(19) United States
(12) Patent Application Publication
(10) Pub. No.: US 2006/0252075 A1
(43) Pub. Date: Nov. 9, 2006

(54) Method for monitoring anti-HIV treatment in HIV infected individuals

(75) Inventors: Daniel Zagury, Paris (FR); Jean-Francois Zagury, Paris (FR)

Correspondence Address: BROWDY AND NEIMARK, P.L.L.C.;624 NINTH STREET, NW, SUITE 300, WASHINGTON, DC, 20001-5303, US

(73) Assignee: NEOVACS, Paris (FR)

(21) Appl. No.: 11/404,115

(22) Filed: Apr. 14, 2006

Related U.S. Application Data
(63) Continuation-in-part of application No. 09/763,369, filed on May 22, 2001, now Pat. No. 7,067,246.
(60) Provisional application No. 60/097,497, filed on Aug. 21, 1998.

(51) Int. Cl. C12Q 1/70; C12Q 1/68

(52) U.S. Cl. 435/6; 435/5

(57) ABSTRACT

The present invention relates to methods of monitoring and improving anti-HIV therapy in a subject. More specifically, the invention discloses that the presence or absence of an anti-Tat neutralizing activity in HIV infected subjects represents a reliable marker allowing the design of appropriate treatment protocols. The present invention also shows that causing or stimulating an anti-Tat neutralizing activity in HIV infected subjects allows to delay treatment resumption.

### CROSS-REFERENCE TO RELATED APPLICATIONS

The present application is a Continuation-in-part of Ser. No. 09/763,369, which is a 371 national stage application of PCT/US99/18770, filed Aug. 20, 1999, which claims benefit of priority to 60/097,497, filed Aug. 21, 1998. The entire contents of these applications are incorporated herein by reference.

### BACKGROUND OF THE INVENTION

1. Field of the Invention

The present invention relates to methods of monitoring and improving anti-HIV therapy in a subject. More specifically, the invention discloses that the presence or absence of an anti-Tat neutralizing activity in HIV infected subjects represents a reliable marker allowing the design of appropriate treatment protocols. The invention also shows that causing or stimulating an anti-Tat neutralizing activity in HIV infected subjects allows to delay treatment resumption.

2. Description of the Related Art

Following immune activation, HIV-1-infected CD4⁺ cells synthesize viral proteins and release virions (Zagury et al, 1986). In infected cells, the regulatory transactivating-transcription protein tat, which is not found in the viral particle, but is encoded by the HIV-1 genome, enhances the transcription of viral mRNA through interaction with the cis-acting RNA sequence termed TAR (Cullen, 1991). In contrast to its enhancing effect on viral replication, tat impairs the normal physiologic machinery of infected T cells, thus contributing to their premature death (Zagury et al, 1986). The tat-induced cellular alterations may also be exercised in uninfected T cells, since this protein, released from acutely infected cells into the extracellular compartment (Ensoli et al, 1993), can target uninfected cells by a mechanism involving interaction with integrins (Hynes, 1992) or other cell membrane receptors (Weeks et al, 1993). Immune cells, which carry a large density of integrin receptors, particularly after activation (Hynes, 1987), may be dysregulated by extracellular tat, as shown in vitro (Zagury et al, 1996). Pretreatment of activated PBMCs with tat induces both T cell immune suppression (Viscidi et al, 1995; Chirmule et al, 1995) and apoptosis (Li et al, 1995; Westendorp et al, 1995; Katsikis et al, 1997). Extracellular tat acts not only on T cells but also on antigen processing cells (APCs), e.g., monocytes, macrophages and dendritic cells (Zagury et al, 1998). The tat-induced immunosuppressive effect is further promulgated by the enhanced secretion of the immunosuppressive cytokine, IFNα, by APCs (Zagury et al, 1998). The present inventors showed that PHA-activated PBMC cultures treated with HIV-1, but not untreated cultures, generate CD8⁺ suppressive T cells. In this in vitro experimental model, the potent role of tat and IFNα in HIV-1-induced immune-suppression was confirmed since specific anti-tat and anti-IFNα, but not control antibodies prevented the generation of CD8⁺ suppressive T cells (Zagury et al, 1998).

Currently, the prognostic indicator or marker is viral load. However, the work of the present inventors shows that it is not a good prognostic indicator of future disease progression at an early stage of disease when the viral load remains low, but is merely an indicator of the stage of disease (asymptomatic versus sick). While a test for the viral p24 protein is commercially available, this test is only used to determine whether an individual is infected, and not as a prognostic indicator/marker of disease progression.

The HIV-1 trans-activating transcriptional (Tat) protein is a key regulatory protein catalyzing proviral transcription and DNA chain elongation in a complex with several cellular proteins (Okamoto et al., 1990; and Ensoli et al., 1993). In addition, Tat is released by infected CD4 cells in the microenvironment of acutely infected lymphoid foci (Marcuzzi et al., 1992; Zauli et al., 1992; and Zhang et al., 1995), and, in its extracellular location, is rapidly taken up by uninfected cells (Marcuzzi et al., 1992; Gutheil et al., 1994; Westendorp et al., 1994; and Wong-Staal et al., 1992). Extracellular Tat acts as a viral toxin to infected cells, enhancing viral production (Re et al., 1995) and, in addition, has effects on uninfected stromal cells (Gallo, 1999; and Ensoli et al., 1990). Its deleterious effects on uninfected immune cells leads to diminished T cell proliferation (Zagury et al., 1998; and Viscidi et al., 1989) and apoptosis (Gougeon, 2003; and Westendorp et al., 1995).

In vitro studies demonstrate antibody directed against Tat is capable of neutralizing the biological effects of Tat(ref). The serum of HIV-1 infected patients contains anti-Tat antibodies, though most often at low levels, likely as a response to the presence of the Tat protein in the extracellular lymph milieu. Furthermore, circulating Abs to Tat are found at higher titer in HIV infected patients with long term non-progression of their disease (Zagury et al., 1998; Re et al., 2001; and Rezza et al., 2005), whereas anti-Tat Abs were inversely correlated with HIV-1 p24 antigenemia (Zagury et al., 1998).

Citation of any document herein is not intended as an admission that such document is pertinent prior art, or considered material to the patentability of any claim in the present application. Any statement as to content or a date of any document is based on the information available to applicants at the time of filing and does not constitute an admission as to the correctness of such a statement.

### SUMMARY OF THE INVENTION

An object of the present invention resides in a method of monitoring anti-HIV treatment in a subject infected with HIV, the method involving assessing the level of anti-tat antibody in a sample derived from said subject, such a level being indicative of the required treatment protocol.

Another object of the present invention resides in a method of monitoring anti-HIV treatment in a subject infected with HIV, the method involving assessing the level of neutralizing anti-tat activity in a sample derived from said subject, such a level being indicative of the required treatment protocol.

In particular embodiments of the method, the absence of anti-tat antibody or neutralizing anti-tat activity in said subject is an indication that the anti-HIV treatment shall be maintained or resumed, and/or the presence of anti-tat antibody or a neutralizing anti-tat activity in said subject is an indication that the treatment may be interrupted or that the treatment interruption may be prolonged.

Yet another object of the present invention resides in a method of monitoring anti-HIV treatment in a HIV subject under structured treatment interruption, the method involving assessing the level of anti-tat antibody in a sample derived from said subject, wherein the presence of a significant anti-tat antibody level in said subject is an indication that the treatment interruption may be prolonged.

A further object of the present invention resides in a method of monitoring anti-HIV treatment in a HIV subject under structured treatment interruption, the method involving assessing the level of neutralizing anti-tat activity in a sample derived from said subject, wherein the presence of a significant neutralizing anti-tat activity in said subject is an indication that the treatment interruption may be prolonged.

A still further object of the present invention resides in a method of monitoring anti-HIV treatment in a HIV subject under structured treatment interruption, the method involving assessing the level of anti-tat antibody in a sample derived from said subject, wherein the absence of a significant anti-tat antibody level in said subject is an indication that the treatment may be resumed.

A still yet further object of the present invention resides in a method of monitoring anti-HIV treatment in a HIV subject under structured treatment interruption, the method involving assessing the level of neutralizing anti-tat activity in a sample derived from said subject, wherein the absence of significant neutralizing anti-tat activity in said subject is an indication that the treatment may be resumed.

Another aspect of the present invention is a method of monitoring anti-HIV treatment in a subject infected with HIV, the method comprising:
(i) assessing the presence of anti-tat antibodies in a sample derived from said subject, and
(ii) assessing the presence of a neutralizing anti-tat activity in a sample derived from said subject having anti-tat antibodies,
   the presence of a neutralizing anti-tat activity being indicative of the required treatment protocol.

A still yet another object of the present invention resides in a method of monitoring anti-HIV treatment in a HIV subject under structured treatment interruption, the method comprising:
(i) assessing the presence of anti-tat antibodies in a sample derived from said subject, and
(ii) assessing the presence of a neutralizing anti-tat activity in a sample derived from said subject having anti-tat antibodies,
   the presence of a neutralizing anti-tat activity being an indication that the treatment interruption may be prolonged.

In the above method, the anti-HIV treatment in said subject having a neutralizing anti-tat activity may be subsequently monitored by assessing the presence of anti-tat antibodies in a sample derived from said subject. Indeed, once the presence of a neutralizing activity has been established, which typically results from the presence of neutralizing anti-tat antibodies, the status of the patient may be further monitored by assessing only the level of antibodies.

In this respect, a further aspect of the present invention is a method of monitoring anti-HIV treatment in a HIV subject under structured treatment interruption, wherein the subject has been previously shown to have a neutralizing anti-tat activity, the method involving assessing the presence of anti-tat antibodies in a sample derived from said subject, wherein the presence of anti-tat antibodies in said subject is an indication that the treatment interruption may be prolonged.

According to a specific embodiment of the present methods, the anti-HIV treatment includes an anti-retroviral therapy, particularly a chemotherapy, preferably HAART.

Furthermore, in a preferred embodiment, the subjects have received an anti-Tat vaccine, in addition to said anti-HIV treatment, particularly a Tat Toxoid.

The sample is typically a sample of serum or blood of said subject, which may be obtained by techniques known in the art. The sample may be treated prior to use, e.g., by dilution, concentration, separation, etc. The term “derived from” indicates the sample is obtained from the patient (or from a sample collection) and, optionally, further treated, e.g., as discussed above.

In a particular embodiment, the level of antibody or neutralizing anti-tat activity measured with the patient sample is compared to a mean value or to the level of antibody or neutralizing anti-tat activity of a reference serum or blood sample. In this regard, a “significant” anti-tat antibody level or neutralizing anti-tat activity is a level or activity, respectively, that is at least twice superior to the mean value or reference sample. More preferably, a “significant” anti-tat antibody level is a level that is at least three, four or more preferably five times superior to the mean value or to the level of the reference sample.

The level of anti-tat antibody can be measured by any method known per se in the art, such as immunological or immuno-enzymatic assays. Typical assays include ELISA, RIA, etc. In a preferred embodiment, the level of anti-tat antibody is measured by ELISA.

The level of neutralizing anti-tat activity is preferably measured by contacting a tat-responsive reporter gene in the presence of Tat and the sample from the subject, and determining the level of expression of the reporter gene. Preferably, the method further includes comparing the level of expression of the reporter gene as measured with the test sample, to the level of expression of the reporter gene as measured in the absence of such test sample and/or in the presence of a reference sample.

The above method may be performed on any HIV subject, typically at different times.

A further object of the present invention is a method of prolonging anti-HIV treatment interruption in a HIV subject, the method comprising administering to the subject an anti-Tat vaccine.

### BRIEF DESCRIPTION OF THE DRAWINGS

FIG. 1 shows a comparison of IFNα titers in sera from Non/Slow Progressor (NP), who are asymptomatic patients, and Fast Progressor (FP), who are sick patients. In the abscissa, IFNα titers are expressed as I.U. (<4, 4-30 and >30), and in the ordinate, percentages of patients NP (□) and FP (▪).

FIGS. 2A and 2B show pictorial representation of p24 OD distribution for NP and Progressed Non-Progressors (NP-P) subpopulations (FIG. 2A) and boxplot representation of the distribution (FIG. 2B). The dark boxes in the boxplots represent 75% of the distribution, the whiskers represent the upper and lower bounds of the outlier thresholds, above and below which are the outliers and the extremes.

FIGS. 3A and 3B show pictorial representation of anti-tat Ab distribution for NP and NP-P subpopulations (FIG. 3A) and boxplot representation of the distribution (FIG. 3B). The dark boxes in the boxplots represent 75% of the distribution, the whiskers represent the upper and lower bounds of the outlier thresholds, above and below which are the outliers and the extremes.

FIGS. 4A-4F show scatterplots of anti-tat Ab levels (O.D.) versus: p24 antigen level (pg/ml) (p<0.001) (FIG. 4A); log₁₀ viral load (RNA copies/ml) (p=Not Significant or NS) (FIG. 4B); CD4 cell count (cells/mm³) (p=NS) (FIG. 4C); anti-p24 Ab level (O.D.) (p=NS) (FIG. 4D); anti-nef Ab level (O.D.) (p=NS) (FIG. 4E); anti-TT Ab level (O.D.) (p=NS) (FIG. 4F) in sera of NP cases who remained stable (×) or progressed (□) during the 21-month follow-up period. Dotted lines correspond to the median. Values in parentheses represent p-values, i.e., significance values obtained from Pearson correlation analyses.

FIGS. 5A and 5B show the kinetics of anti-Tat antibody (Ab) production (FIG. 5A), as measured by enzyme-linked immunosorbant assay (ELISA), and a histogram of anti-Tat antibody titers in the sera collected 4 to 6 months after the first injection (FIG. 5B). In FIG. 5A, sera were used at 1:500 dilution and antibody levels were expressed as optical density values (O.D.). Sera from nonimmunized study subjects did not exhibit detectable levels of Tat antibodies. A, B, C, D, and E represent immunized study subjects. In FIG. 5B, Tat antibodies were tested in sera at 1:1,000 to 1:64,000 dilutions and measured by ELISA. Titers are expressed as the highest dilution giving a positive reaction above threefold preimmunizing levels. Sera from nonimmunized individuals (controls: F and G) did not exhibit detectable titers of antibodies.

FIG. 6 is a schematic for the design of the 2 year structured treatment interruption study (2002-2004) which followed sanofi-pasteur Phase I/II anti-Tat vaccine trial.

FIG. 7 shows Kaplan-Meier correlation between Ab responders (▬) versus Ab non responders ( - - - ) and onset of HAART as a function of time (1) in patients from the 3 groups (Total cohort); (2) in patients with an established chronic infection from the 3 groups; (3) in patients from DcChol cohort (group 1 and 2), and (4) in patients with chronic infection from DcChol cohort (◯: censored patients).

FIGS. 8A and 8B are graphs showing anti-Tat immune reaction of patients from the DcChol cohort (n=20) at their enrollment in Sanofi-Pasteur vaccine trial (t₀) (white columns) and at onset of ATI (day 1) (black columns). HAART resumption is indicated as (*). In FIG. 8A, the serum Ab titers are in μg per ml. Cut off of positivity (10 μg) corresponded to 5 fold reference serum (2 μg) (T=Tat Toxoid and T+DC=adjuvanted Tat Toxoid vaccinees). In FIG. 8B, the Tat Inhibitory capacity (ETIC) score of patients serum at day 1 compared to the reference serum value (see Example 4, Materials and Methods).

FIGS. 9A and 9B are graphs showing Tat-inhibitory capacity (ETIC) of IgG purified through protein G chromatography. β-galactosidase assay was carried out after preincubating Tat protein with purified IgG from sera (day 1) at various concentrations. In FIG. 9A, IgG ETIC from 3 patients (AB who received HAART, XY and WZ Ab responders who remained HAART-free). IgG doses ranging from 15-500 μg permitted determining the quantity of these Abs inhibiting 50% of Tat transcriptional activity (Inhibitory Index 50, II₅₀) in a β-galactosidase assay carried out using Tat protein at 50 ng/ml. In this test, II₅₀ from Ab, XY, WZ serum at day 0 were 250 μg, 96 μg and 48 μg respectively while II₅₀ values from reference serum were over 500 μg. In FIG. 9B, II₅₀ of purified IgG from patients of DcChol cohort at day 1 of ATI obtained in a β-galactosidase assay using Tat protein at a concentration of 30 ng/ml.

### DETAILED DESCRIPTION OF THE INVENTION

To investigate whether particular serum markers in HIV infected individuals, such as antibodies against HIV-1 specificities or the p24 viral marker, are predictive of disease progression, serum was collected from 104 HIV-1 infected non-disease progressing individuals (chosen as individuals who have been infected more than eight years with CD4 cell counts always about 500 per mm³ and with no clinical sign of disease) at the time of enrollment in the study and at follow-up one to two years later. At the follow-up, 26 of the HIV-1 infected individuals showed signs of disease progression (CD4 cell decrease over 30% or presence of clinical symptoms). In this study, the present inventors discovered that, of the serum markers tested (antibodies to HIV antigens including env, gag, nef and tat proteins, as well as p24 antigenemia, viremia, CD4 cell count and IFNα titer), anti-tat antibodies and p24 antigenemia were the best predictive markers in the serum, with the anti-tat antibodies being inversely correlated with p24 antigenemia, which is already known as a marker of viral replication. These were the only two serum markers that significantly correlated the two subgroups, infected individuals with serum marker level above the median and subjects with serum marker level below the median, with progression and non-progression of disease.

The serum levels of anti-tat antibodies and p24 protein are used as prognostic markers. By “prognosis”, it is intended that the progressive state of the HIV infected individual in early infection can be predicted. Thus, as the serum levels of anti-tat antibodies correlate inversely with disease progression, at high serum levels of anti-tat antibodies, the HIV infected individual is in a state of non-progression. The serum level of anti-tat antibodies determines the progression state of an HIV infected individual. As long as the anti-tat antibody levels are still high, the individual is non-progressing. Once the level of anti-tat antibodies begins to drop, then the prognosis is that the infected individual is entering a progressive state.

While the serum level of anti-tat antibodies is the preferred prognostic marker, the serum level of tat protein can also be used as well (complexed or uncomplexed). Like the correlation between anti-tat antibodies and p24 protein, the serum level of tat protein correlates inversely with anti-tat antibodies. The higher the serum level of anti-tat antibodies, the lower the serum level of tat protein, and, conversely, the lower the serum level of anti-tat antibodies, the higher the serum level of tat protein.

In addition, besides measuring the serum level of anti-tat antibodies or tat protein as a prognostic marker/indicator, the serum level of p24, which is already a known marker for viral replication, can also be used as a prognostic marker in the present invention because it has been discovered by the present inventors to be highly correlative with disease progression. Tests for p24 to diagnose HIV-1 infection (but not the progressive state of the individual) are commercially available.

It is further contemplated that the measurement of the level of one or more of the anti-tat antibody, tat protein or p24 protein prognostic markers can be combined with the measurement of other markers, such as viral load, CD4 cell count, or IFNα, to provide a more complete determination of the overall status of the HIV-1 infected individual.

The level of any of the serum markers discussed above can be measured by any of a variety of assay techniques and is not limited to ELISA used in the Examples hereinbelow, as would be appreciated by those of skill in the art. For instance these techniques could include the use of blots, beads, plates, immunoprecipitations, monoclonal or polyclonal antibodies against tat protein or fragments, the tat protein or tat peptides, competitive measurements or direct assays, enzymatic or radioactive markers, etc.

The correlation of high serum levels of anti-tat antibodies with non-disease progression among HIV infected individuals and low levels with disease progression strongly suggest that raising the serum level of anti-tat antibodies through active immunization may control viral replication. The serum level of anti-tat antibodies is monitored over time to determine whether the level of anti-tat antibodies has dropped sufficiently or the level of tat protein has increased sufficiently to warrant administration of a tat vaccine. By active immunization with a tat vaccine, the level of anti-tat antibodies is brought back up to a level indicative of non-disease progression. This same individual is monitored over time to confirm that the serum level of anti-tat antibodies is at a level predictive of non-disease progression and that this level is maintained over time. If a drop in serum level of anti-tat antibodies or an increase of tat protein in the serum is observed, further intervention, such as additional immunization with a tat vaccine, can be contemplated.

While the preferred tat vaccine is the tat toxoid vaccine disclosed in WO 96/27389, which contents are herein incorporated entirely by reference, the tat vaccine used in accordance with the present invention is not limited to such a tat toxoid vaccine. As will be appreciated by those skilled in the art, any tat vaccine which achieves the intended purpose of stimulating an immune response and raise the titer of anti-tat antibodies is within the skill of the art to use.

Tat vaccine experiments were performed in macaques (Cafaro et al., 1999; Pauza et al., 2000; Voss et al., 2003; Osterhaus et al., 1999; and Liang et al., 2005) to evaluate whether induction of neutralizing Abs inhibiting extracellular Tat (Table Ia-B) or cellular immunity to Tat triggering lysis of infected cells (Table Ia-A, C, D, E) would control SHIV infection. Following viral challenge, none of the immunizations generated sterilizing immunity but most led to a marked decrease of viremia (Table Ia).

TABLE Ia
Effects of anti-Tat vaccine in non human primate

| | | | | Principal | | |
|---|---|---|---|---|---|---|
| | | Anti Tat immune | | investigator | | |
| | Vaccine | target | Source | (Institution) | Macaques | Safety |
| A | Biologically | Cellular response | ABL, | B. Ensoli | 7 Healthy | +++ |
| | active Tat | for lysis of infected | Bethesda | (ISS)⁶ | Macaques | |
| | | cells | | | (Preventive) | |
| B | Tat Toxoid in | Ab neutralization | Neovacs | D. Pauza | 6 Healthy | +++ |
| | IFA¹ | of extracellular Tat | Paris | (Univ. of | Macaques | |
| | | | | Wisconsin) | | |
| C | Fusion Tat- | Ab and cellular | GSK⁵ | G. Voss | Healthy | +++ |
| | Nef + gp120 in | response for | Rixensart | GSK | Macaques | |
| | AS2OA² | minimal virus | | Rixensart | | |
| | | replication | | | | |
| D | nef + tat DNA | Cellular response | Erasmus | A. Osterhaus | 2 Macaques | +++ |
| | in MVA | for lysis of infected | MC | Erasmus MC | | |
| | vector³ | cells | Rotterdam | Rotterdam | | |
| E | tat in Ad/5 | Cellular response | Merck | X. Liang, J. W Shiver | 4 Macaques | +++ |
| | vector⁴ | for lysis of infected | West-Point | Merck Research | | |
| | | cells | | Laboratories | | |

| | | | | Efficacy | | |
|---|---|---|---|---|---|---|
| | Immunogenicity to Tat | | | post | | |
| | Cellular | Ab | | challenge | | |
| | response | Titer | Neutralizing Abs | with SHIV | Observation | References |
| A | ++ | + | 0 | Viremia ↓ | Partial protection | Cafaro et al., 1999 |
| | | | | Partial | not correlated with | |
| | | | | protection | Ab response | |
| B | ++ | +++ | ++ | Viremia ↓ | Sterilizing | Pauza et al., 2000 |
| | | | | | immunity | |
| | | | | | not achieved | |
| C | ++ | ++ | NA | Viremia ↓ | Sterilizing | Voss et al., 2003 |
| | | | | Prevention | immunity | |
| | | | | of AIDS for | not achieved | |
| | | | | >2.5 years | | |
| D | +++ | NA | NA | Viremia ↓ | Sterilizing | Osterhaus et al., |
| | | | | | immunity not | 1999 |
| | | | | | achieved | |
| E | +++ | Low | 0 | None | Cellular | Liang et al., 2005 |
| | | negligible | | | immunization with | |
| | | | | | negligible Ab | |
| | | | | | uneffective | |

¹IFA: Incomplete Freund adjuvant constituted by Montanide ISA51 from Seppic (Paris), also known as Salk adjuvant.
²AS02A is a GSK adjuvant constituted by MPLA in QuilA;
³MVA: Murine vaccine Ankara vector
⁴Ad/5: Adenovirus type 5
⁵GSK: Glaxo Smith Kline
⁶ISS: Institute Superior Sanitat (Roma)

These data prompted the present inventors and others to test a therapeutic Tat vaccine in humans (Gringeri et al., 1998 and 1999; Hermans et al., 2003; Caputo et al., 2004; http//chi.ucs2.edu/vaccines; and Calarota et al., 1999) (Table Ib).

TABLE Ib
Phase I Clinical trials of anti-Tat vaccines

| | | | Principal | | | |
|---|---|---|---|---|---|---|
| | | | investigator | | | |
| | Vaccine | Source | (Institution) | Stage | Subjects | Aim |
| A | Tat Toxoid | Neovacs | A. Gringeri | Phase I | 6 Healthy individuals | Safety and anti- |
| | in IFA | Paris | (Univ. Hosp. | | | Tat |
| | | | Maggiore) Milan | | | neutralizing |
| | | | 1998 | | | Abs |
| | Tat Toxoid | Neovacs | A. Gringeri | Phase I | 14 Infected subjects | Safety and anti- |
| | in IFA¹ | Paris | (Univ. Hosp. | | (chemotherapy + or −) | Tat |
| | | | Maggiore) Milan | | | neutralizing |
| | | | 1998 | | | Abs |
| B | Tat toxoid in | Sanofi- | R. Redfield | Double | 32 HIV-1 patients | Neutralization |
| | Alum | pasteur | (IHV Baltimore) | blind | (HAART pre- | of Tat Toxin |
| | | | 2002 | Phase I/II | treatment) | |
| | Tat toxoid in | Sanofi- | N. Clumeck | Double | 32 HIV-1 patients | Neutralization |
| | DcChol | pasteur | (Hosp. St Pierre | blind | (HAART pre- | of Tat Toxin |
| | | | Brussels) | Phase I/II | treatment) | |
| | | | 2002 | | | |
| C | Biologically | Chiron | B. Ensoli | Double | 50 HIV-1 negative | Enhancement |
| | active Tat² ± Alum | | (ISS Roma and | blind | individuals | of TH1 |
| | | | Ouganda) | Phase I | | response and |
| | | | 2003/2004 | trial | | CTL |
| D³ | Fusion Tat- | GSK | NA | Phase I | NA | Preventive |
| | Nef + gp120 | | | | | |
| | in AS02A | | | | | |
| E | Tat DNA | Karolinska | S. Calarota; | Phase I | 2 naive + HAART | Safety |
| | | Institute | B. Warren | | patients | immunogenicity |
| | | | (Karolinska | | | |
| | | | Institute) | | | |

| | | Immunogenicity to Tat | | | |
|---|---|---|---|---|---|
| | | | | Serum | |
| | | Cellular | | Neutralizing | |
| | Safety | response | Ab Titer | Capacity | References |
| A | No | Present | +++ | High | Gringeri et al., 1999 |
| | side | | | | |
| | effects | | | | |
| | No | ″ | ++ | High | Gringeri et al., 1998 |
| | side | | | | |
| | effects | | | | |
| B | No | Negligible | ++ | NA | Hermans et al., 2003 |
| | side | | | | |
| | effects | | | | |
| | No | Present | +++ | +++: | Hermans et al., 2003 |
| | side | | | in 6/12⁴ | |
| | effects | | | | |
| C | NA | NA | NA | NA | Caputo et al., 2004 |
| D³ | NA | NA | NA | NA | http//chi.ucs2.edu/vaccinees |
| E | No | Proliferation | ± | NA | (Calarota et al., |
| | side | | | | 1999) |
| | effects | | | | |

¹IFA: Incomplete Freund adjuvant constituted by Montanide ISA51 from Seppic (France), known as Salk adjuvant.
²No report relevant to the results of the trial are at this date available (NA)
³No information yet available on the trial under evaluation conducted by GSK (Glaxo Smith Kline)
⁴High Ab response (titer and neutralization) in responders (50%) of Tat Toxoid immunized subjects.
NA: Not available

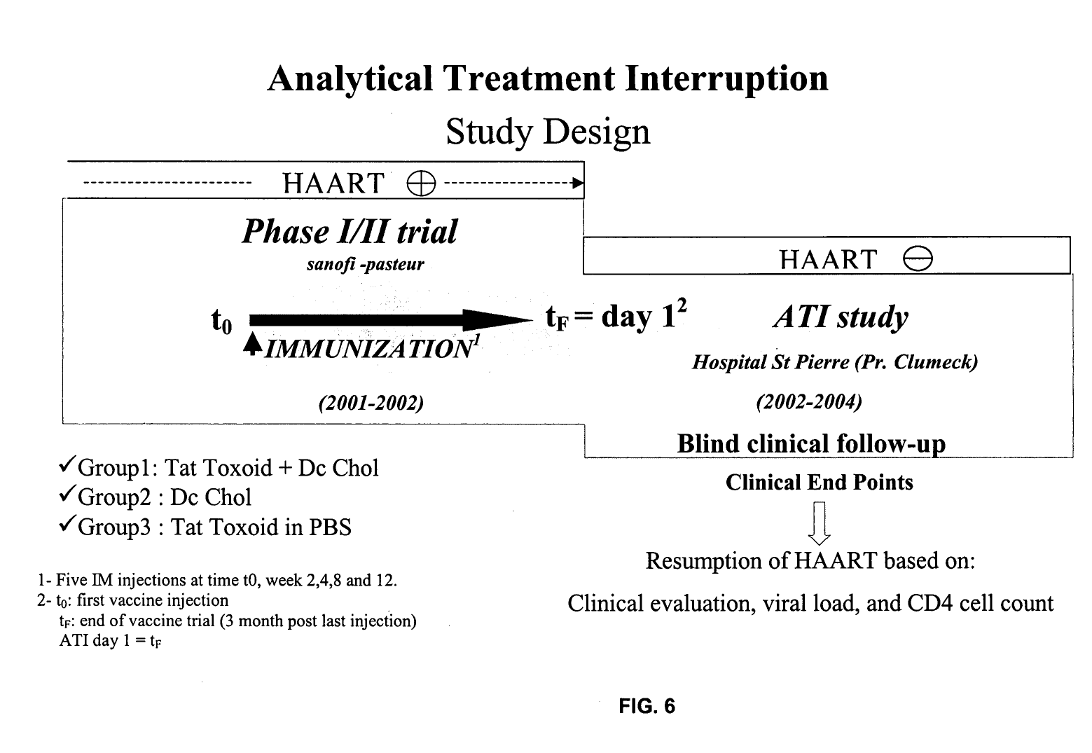

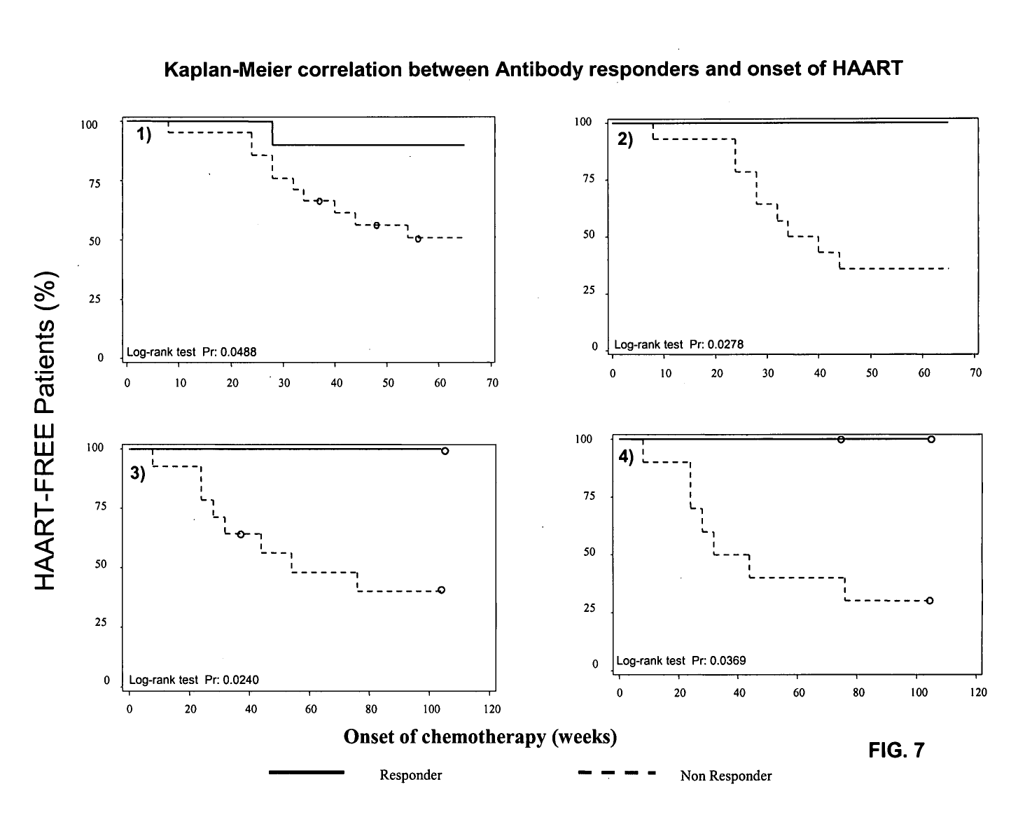

In 2002, a Phase I/II, placebo controlled trial of a candidate Tat Toxoid immunogen adjuvanted with DcChol and produced by Sanofi-Pasteur was conducted. The subsequent clinical outcome of the 31 patients enrolled in the trial who, following vaccination, discontinued their antiretroviral (ARV) regimens as part of an observational 2 year structured treatment interruption (STI) study (FIG. 6) is reported in Example 4 below. Vaccinees were blindly monitored by their own treating physician, regardless of the Tat vaccine treatment arm. Patients sera reactivity to Tat followed up through STI emphasized the role of Tat Toxoid induced anti-Tat Abs in their clinical outcome.

The results presented in Example 4 show that the development of positive Tat antibody titers in the subject was followed by a prolonged delay of anti-retroviral therapy resumption. These results also show that the absence of anti-Tat neutralizing activity in the subjects always preceded or was concomitant with anti-retroviral therapy resumption.

Having now generally described the invention, the same will be more readily understood through reference of the following examples, which are provided by way of illustration and is not intended to be limiting of the present invention.

### EXAMPLE 1

### Materials and Methods

Patients

Two groups of genetically homogeneous French Caucasians were recruited in various hospital centers in Paris and surrounding provinces in the context of the GRIV program (Hendel et al, 1996; Rappaport et al, 1997). The first group was composed of Non/Slow Progressors (NP) and consisted of 182 individuals enrolled on the basis of the following criteria: seropositivity for over 8 years (mean and median both 12 years); clinically asymptomatic in the absence of antiretroviral therapy; CD4 cell count never below 500/mm³. These subjects represented approximately 1% of the seropositive individuals attending the hospital centers. In this group of asymptomatic NPs, 104 individuals had their follow-up available for a period of 1-2 years (mean and median 20 and 21 months) after their initial enrollment: clinical symptoms (CDC Stage B or C as defined by the 1993 classification) and laboratory parameters including CD4 cell count. Twenty-six of these 104 NPs showed signs of progression during 1 to 2 years of follow-up and were called Progressed Non-Progressors (NP-P), while the remaining 78 were called Non-Progressed Non-Progressors (NP-NP).

The second group was composed of 67 fast progressors (FP) defined as follows: seropositivity<3 years, treated with anti-HIV-1 drugs, clinically asymptomatic or not, and CD4 cell count below 300/mm³. The FP population represented about 5% of the seropositive individuals recruited in clinics.

Upon enrollment in the GRIV study, cells and serum were shipped overnight to the laboratory of the present inventors and stored frozen. The present work was performed on this bank of sera collected at enrollment in the study.

Serological Tests

Sera from NP and FP patients, collected at the time of enrollment, and of healthy seronegative controls were investigated for the following serological parameters:

(1) The titer of antibodies to HIV-1 p24 protein and gag peptides, tat protein and tat peptides, nef protein, gp160 protein and gp120 peptides and tetanus toxoid (TT) was determined by standard ELISA, and results were expressed as OD of test serum subtracting the mean OD of control sera. The gag, env, tat, and nef proteins were prepared by recombinant engineering. The peptides tested were chemically synthesized (Neosystem, Strasbourg, France).

(2) Viral load in the serum was assessed using the Virionquant kit (Paris, France) which gives results as a number of eq copies/mm³. Briefly, the viral RNA is extracted from the serum and used for RT-PCR with multiple pairs of HIV-1 primers. The amplified DNA product is then detected and quantitated using an ELISA system. The ODs are compared with those obtained for a standard, VirionStandardj, covering 4 logs of eq copies/ml (from 10² to 10⁵) and treated in parallel. The statistical analysis was performed on the log values.

(3) p24 antigenemia was measured by the HIV-1 p24 core profile ELISA kit (NEN-Dupont, cat# NEK060B) with the results expressed in pg/ml.

(4) IFNα Levels: The biological assay using VSV and MDBK cells was utilized to determine IFNα levels in serum, and the titers compared to the standard NIH (2.5×10⁶ IU/mg protein) were expressed in I.U.

Statistical Analysis

Statistical analyses were performed using SPSS for Windows, Ver. 7.5. Cumulative frequency distributions on each biologic parameter for all cases allowed for dichotomization of all continuous variables, in preparation for Chi-square analyses. Student T-tests were performed on each biologic parameter to compare means and standard deviations of values for NP-NP and NP-P groups. Pearson Correlations coefficients were computed to examine linear, one-to-one relationships for each pair of biologic parameters. Univariate linear regression analyses were conducted to examine the independent contribution of each covariate to the progression status (Table 5). Multivariate regression analyses were performed to examine the relative behavior of each covariate in the presence all covariates in predicting progression.

### Results

Tables 1 and 6 present data comparing NP and FP groups. Tables 2 to 5 present statistical comparisons of NP subjects on whom follow-up data were available (104 individuals): 78 of these NP cases were considered to be clinically stable (subgroup NP-NP); and 26 exhibited signs of progression towards AIDS during the follow-up (subgroup NP-P). Criteria, such as sex, age, as well as other HIV-1 antibody specificities, including gp160 and gp120 peptides (V3 loop and CD4 binding region), are not reported here because they do not exhibit any significant information.

Biological Characteristics Correlated to NP and FP Groups

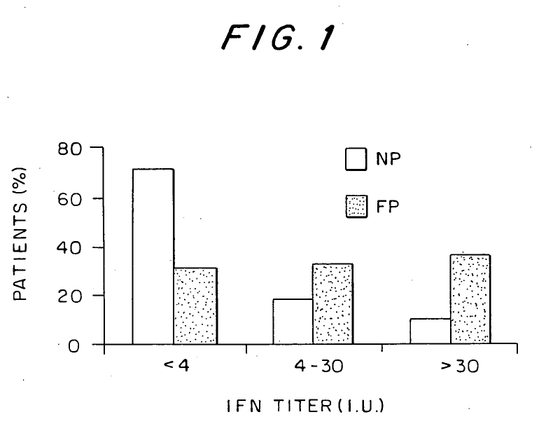

In addition to different predisposing genetic traits (Li et al, 1995; Westendorp et al, 1995), NP and FP populations of the GRIV cohort exhibited quantitative differences in serologic parameters (Table 1). In particular, the mean anti-p24 antibody level was significantly higher in the sera of NP individuals (p<0.0001) relative to the levels in sera from FP patients. The mean IFNα titers were significantly higher (p<0.0001) in the serum of FP than in those of NP. FIG. 1 shows the individuals with abnormal levels of serum IFNα. In addition, serum p24 antigen levels were significantly higher (p=0.004) and tat antibodies lower (p=0.03) in the FP group than in the NP group, but to a lesser extent than anti-p24 and IFNα. In contrast, the means of other tested parameters, including nef and TT antibody levels, were not significantly different in the two groups of patients (Table 1).

TABLE 1
T-test Comparisons of the Mean Biologic
Parameters in the GRIV Cohort

| | | | p-value |
|---|---|---|---|
| Biological | NP Mean (S.D.) | FP Mean (S.D.) | (generated from |
| Parameter | N = 182 | N = 67 | Student T-test) |
| p24 antibody | 1.70 (0.72) | 0.56 (0.45) | p < 0.0001 |
| (O.D.) | | | |
| IFNα titer (IU) | 6.9 (13.6) | 22.6 (22.2) | p < 0.0001 |
| p24 ag (pg/ml) | 21.22 (16.4) | 29.55 (28.6) | p = .0046 |
| tat antibody | 0.39 (0.25) | 0.32 (0.17) | p = .032 |
| (O.D.) | | | |
| nef antibody | 0.22 (0.25) | 0.20 (0.26) | p = NS |
| (O.D.) | | | |
| TT antibody | 0.71 (0.45) | 0.82 (0.46) | p = NS |
| (O.D.) | | | |

NS = not significant

Biological Characteristics Associated with Progression of HIV-1 Infection in the NP Population

Among the 104 NP patients for whom the follow-up was available since their enrollment, 26 subsequently exhibited a decline in CD4⁺ cell count below the initial value of greater than 30% or, alternatively, exhibited signs of clinical illness corresponding to CDC Stage B or C as defined by the 1993 classification. They were designated Progressed Non-Progressors (NP-P), while the remaining 78 stable individuals were designated Non-Progressed Non-Progressors (NP-NP).

Summary analyses were performed on each clinical and biologic parameter to explore their distribution and scatter. Once the ranges of values were found to be somewhat normally distributed and the means and medians determined, each parameter was divided into “High” and “Low” (or above and below their respective medians). Two-by-two tables were constructed using the Chi-square analysis procedure to test observed proportions against expected proportions within the NP and NP-P groups (Table 2).

TABLE 2
Chi-square Analyses of Biologic Parameters vs. Progression Status

| | | N of Cases | | | |
|---|---|---|---|---|---|
| Biologic | | NP—NP | NP - P | | |
| Parameter | Median Split | N = 78 | N = 26 | χ² | p-value |
| Anti-TT | Low (0-.71) | 38 | 13 | 0 | .545 |
| | High (.72+) | 40 | 13 | | |
| Anti-tat | Low (0-.30) | 33 | 19 | 7.38 | .006 |
| | High (.31+) | 45 | 7 | | |
| Anti-nef | Low (0-.14) | 37 | 15 | 0.82 | .365 |
| | High (.15+) | 41 | 11 | | |
| Anti-p24 | Low (0-2.1) | 38 | 13 | 1.03 | .590 |
| | High (2.2+) | 38 | 13 | | |
| p24 ag | Low (0-19) | 44 | 8 | 5.12 | 0.20 |
| | High (20+) | 34 | 18 | | |
| Viral Load | Low (0-3.9) | 41 | 10 | 2.01 | .141 |
| | High (4.0+) | 36 | 16 | | |
| CD4 | Low (0-710) | 34 | 17 | 3.10 | 0.78 |
| | High (711+) | 41 | 9 | | |

NP—NP are stable Non-Progressors while NP - P are the Non-Progressors who have progressed during the follow-up.
“High” and “low” groups were determined by dichotomizing all cases by the median split for each biologic parameter.

Among the biological parameters tested, the most discriminative for stability versus progression was anti-tat antibodies (p=0.006), followed by p24 antigenemia (p=0.020). CD4 cell counts (p=0.078) and viral load (p=0.141) were not as impressive in their ability to discriminate between these progression categories. As shown in the subgroup of NP exhibiting higher CD4⁺ cell counts, there were fewer cases with signs of progression than there were in cases with lower CD4 cells counts (18% versus 33%), but this relationship was not statistically validated. Although the viral load was low in most NP patients (<104 copies/ml), those who later progressed toward AIDS had relatively higher levels of serum viral RNA (31% versus 19%), but this disparity did not achieve statistical significance. No difference between the two groups was found with other antibody specificities including p24, nef and TT.

As seen in Table 3, Pearson correlation analyses were conducted on all 104 NP patients to examine the one-to-one relationships of each pair of continuous biological parameters, and were followed by analyses for each disease-progression category. Anti-tat and p24 (both measured in O.D.) were very highly and inversely correlated (r=−0.641, p=0.000). Interestingly, anti-TT levels were moderately correlated with anti-tat levels (r=0.341, p=0.000). Pearson correlations were also performed within each disease-progression category resulting in very much the same associations; however, no significant correlation was found between viral load and anti-p24, and viral load and anti-TT in the NP-P group.

TABLE 3
Pearson Correlations of Biologic Parameters
for All Cases, and Then by Disease Progression Status

| | | All Cases | Status = NP | Status = NP − P |
|---|---|---|---|---|
| | | n = 104 | n = 78 | n = 26 |
| Correlation Pair | | r (p value) | r (p value) | r (p value) |
| Anti-tat | Anti-TT | .341 (.000) | .310 (.006) | .443 (.024) |
| Anti-tat | p24 | −.641 (.000) | −.602 (.000) | −.732 (.000) |
| p24 | Anti-p24 | −.238 (.016) | −.226 (.050) | −.352 (NS) |
| p24 | Anti-TT | −.335 (.001) | −.253 (.025) | −.623 (.001) |
| VL | Anti-tat | −.176 (NS) | −.202 (NS) | .291 (NS) |
| VL | Anti-p24 | −.204 (.040) | −.272 (.018) | .008 (NS) |
| VL | Anti-TT | −.217 (.028) | −.229 (.045) | −.052 (NS) |

In the NP-P patients, for whom levels of p24 antigenemia were higher than in the NP group (Table 2), tat antibody levels were inversely correlated to p24 antigenemia (r=−0.732, p<0.001) (Table 3). Furthermore, most of the NP-P patients who are at advanced stages of infection exhibited AIDS-related clinical manifestations, despite anti-retroviral therapy. Of the few remaining asymptomatic individuals (20%), the majority belonged to the NP-P subgroup with high tat antibodies (>0.31 O.D.)

Univariate and multivariate analyses of biologic factors and their value in predicting progression status were then performed. When each covariate was examined as a univariate predictor of disease progression (Table 4), anti-tat, p24 and viral load significantly predicted progression status. However, none of these independent relationships reflect the multiplicity of factors acting in consonance to produce disease progression.

TABLE 4
Univariate Analysis of Covariates as Predictors of Disease Progression

| Independent Covariate | β | p-value |
|---|---|---|
| Anti-nef | −.101 | .309 |
| Anti-p24 | .049 | .625 |
| Anti-tat | −.208 | .035 |
| Anti-TT | −.092 | .351 |
| p24 | .225 | .022 |
| VL | .216 | .028 |

A more practical approach, perhaps, to this exploration of interactions among biologic parameters is the multivariate approach. Four methods of stepwise multiple regression were performed to examine the influence of each covariate on disease progression in the presence of all others (Table 5).

TABLE 5
Backward Elimination of Covariates from a Multiple Regression
Model to Predict Disease Progression

| | | Unstandardized | | | | |
|---|---|---|---|---|---|---|
| | | Coefficients | | Standardized | | |
| | | | Std. | Coefficients | | |
| Model | | B | Error | of β | t | Sig. |
| 1 | (Constant) | .495 | .590 | | .840 | .403 |
| | Anti-nef | −.187 | .309 | −.061 | −.607 | .545 |
| | Anti-p24 | .186 | .116 | .155 | 1.605 | .112 |
| | Anti-tat | −.212 | .707 | −.039 | −.300 | .765 |
| | Anti-TT | 7.305E−02 | .205 | .038 | .357 | .722 |
| | p24 | 1.320E−02 | .008 | .229 | 1.711 | .090 |
| | VL | .149 | .073 | .211 | 2.042 | .044 |
| 2 | (Constant) | .387 | .463 | | .835 | .406 |
| | Anti-nef | −.200 | .304 | −.065 | −.659 | .512 |
| | Anti-p24 | .190 | .115 | .169 | 1.652 | .102 |
| | Anti-TT | 6.738E−02 | .203 | .035 | .332 | .740 |
| | p24 | 1.461E−02 | .006 | .253 | 2.399 | .018 |
| | VL | .151 | .072 | .214 | 2.093 | .039 |
| 3 | (Constant) | .474 | .381 | | 1.243 | .217 |
| | Anti-nef | −.205 | .303 | −.067 | −.678 | .499 |
| | Anti-p24 | .186 | .114 | .166 | 1.637 | .105 |
| | p24 | 1.395E−02 | .006 | .242 | 2.432 | .017 |
| | VL | .145 | .070 | .207 | 2.077 | .040 |
| 4 | (Constant) | .406 | .367 | | 1.106 | .272 |
| | Anti-p24 | .179 | .113 | .159 | 1.582 | .117 |
| | p24 | 1.416E−02 | .006 | .246 | 2.483 | .015 |
| | VL | .154 | .069 | .218 | 2.228 | .028 |
| 5 | (Constant) | .804 | .268 | | 2.996 | .003 |
| | p24 | 1.203E−02 | .006 | .209 | 2.153 | .034 |
| | VL | .133 | .069 | .189 | 1.948 | .054 |

a. Dependent Variable: STATUS

The most informative entry method of covariates is the backward elimination model which removes covariates in order of increasing predictive value on progression status. All continuous covariates were entered into the equation to predict progression status (1=Stable NP, 2=Progressed NP). Curiously, the first covariate to be removed from the model was anti-tat, followed by anti-TT, anti-nef, then anti-p24, leaving Viral Load (β=0.133, p=0.054) and p24 (β=0.012, p=0.034) as the most important predictors of the disease progression status.

Each covariate was divided into two groups (high or low) by the respective median of the distribution. These dichotomized covariates were then entered into a multivariate regression to measure their independent contributions in predicting disease status; a surprising reversal of covariate removals was observed, which indicates that p24 (which one would expect to be the most important predictor of disease status) was removed first, indicating that it had the least predictive effect on disease progression, followed by anti-nef, anti-TT, anti-p24, viral load, leaving anti-tat as the most prominent predictor of disease progression (β=−0.465, p=0.007). The discordance of results from one method to another leads one to seriously consider the implications of making analytic decisions about making important grouping decisions for continuous biologic parameters (i.e., dichotomizing covariates before entry into a complex system).

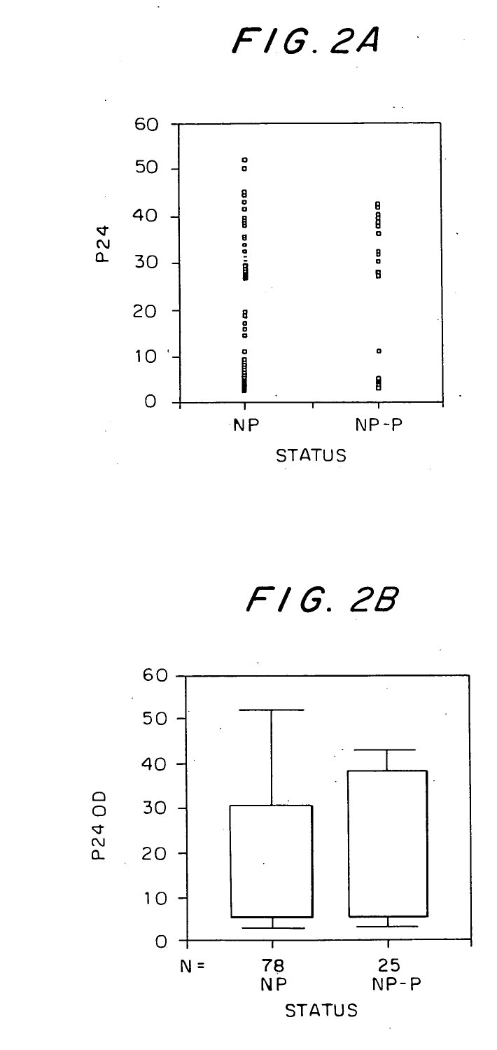

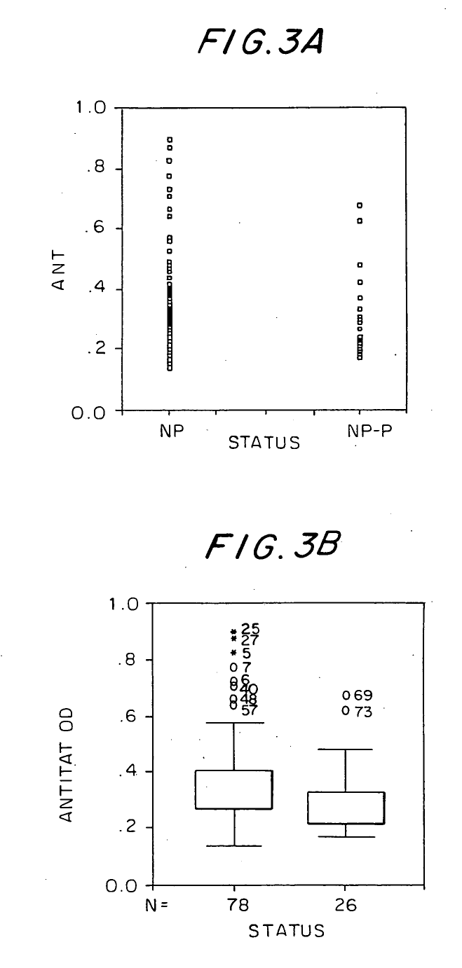

FIGS. 2A-2B and FIGS. 3A-3B illustrate the overall means and scatter of the p24 and the anti-tat values as measured by optical density for the NP-NP and NP-P groups. Relationships between tat Antibody Levels and Other Parameters

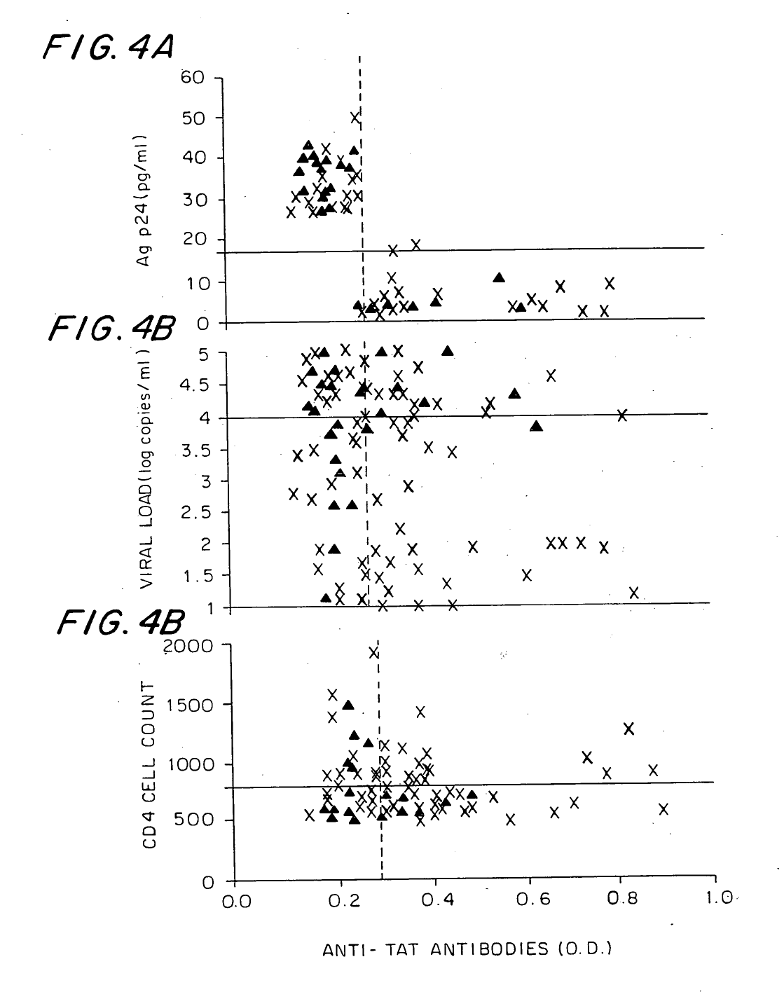

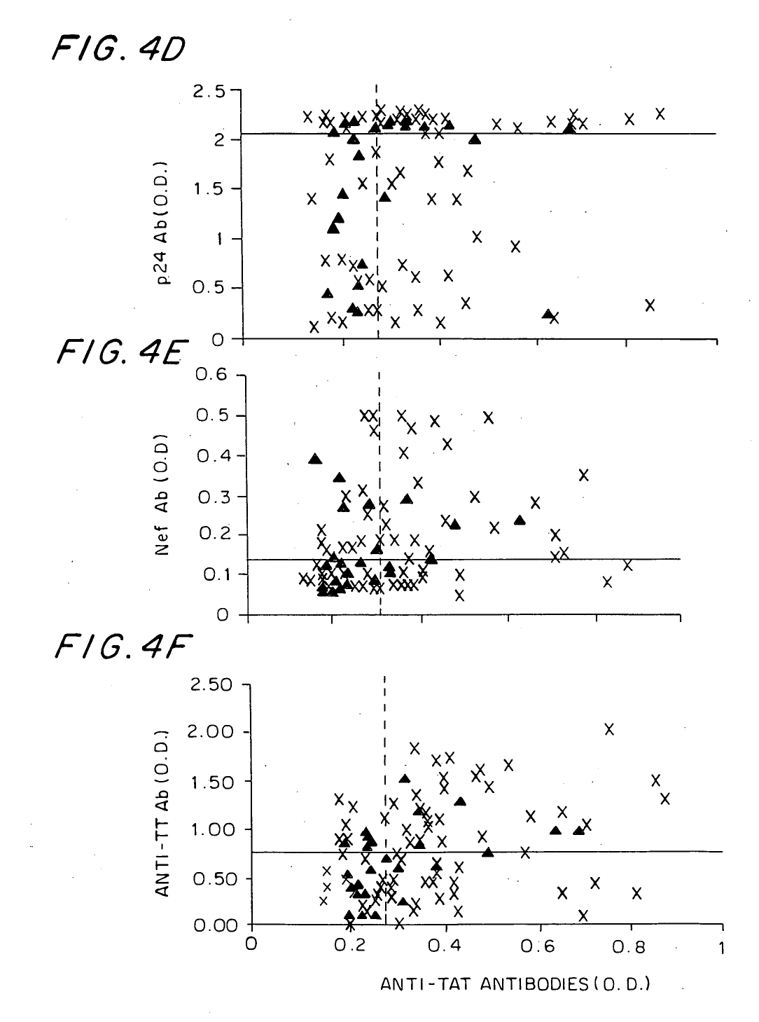

In the 182 NP enrolled in the cohort, tat antibody levels were compared with the other biological parameters to investigate the mechanisms leading to the strong correlation between high tat antibody levels and clinical stability. FIGS. 4A-4F show that in the NP subgroup with high tat antibody levels (>0.31 O.D.; i.e., above the median), fewer subjects exhibited signs of progression than in the subgroup with low tat antibody levels (<0.31 O.D.; i.e., below the median). These differences are highly significant (Table 6).

TABLE 6
T-test Comparison between High and Low anti-tat Responders for
Biological Parameters in NP and Subjects
Mean (SD)

| | NP | | | FP | | |
|---|---|---|---|---|---|---|
| | tat | tat | | tat | tat | |
| | high* | low | p | high* | low | p |
| p24 ag (pg/ml) | 8.03 (7.4) | 35 (10.9) | <.001 | 7.03 (29.2) | 9.97 (23.8) | <.001 |
| p24 antibody (O.D.) | 1.77 (0.7) | 1.64 (0.73) | NS | 0.43 (0.38) | O.66 (0.64) | NS |
| CD4 cells/ml | 793 (295) | 819 (333) | NS | 365 (276) | 326 (223) | NS |
| Lot¹⁰ Viral Load (eq. | 3.36 (1.19) | 3.52 (1.18) | NS | ND | ND | — |
| RNA copies/ml) | | | | | | |

ND = Not Determined
NS = Not significant
For each parameter, patients were distributed into two groups based on tat antibody levels which were high (above the median) and low (below the median).

In Table 6 the subgroups of NP with high and low tat antibody levels were compared with the T test for clinical and laboratory parameters (CD4⁺ cell counts, p24 antigen, p24 antibody, and viral load). P24 antibodies, viral load, and CD4 did not discriminate significantly between NPs with low and high tat antibody levels while p24 antigenemia did. The same result was found comparing FPs with low and high tat antibody levels.

### Discussion

The GRIV project was initiated to investigate genetic and immunologic parameters which determine the disparate courses of disease progression in various HIV-1 infected individuals ranging from 2 to greater than 17 years. To perform the study, two groups of genetically well-defined seropositive subjects were selected, one group was composed of healthy asymptomatic subjects infected for 8 years (NP), and the other group was composed of patients at advanced stages although infected more recently (<3 years) (FP) recruited in different hospital centers in France. The selection criteria used to include subjects in either of these two extreme groups of patients were such that NP represents about 1% and FP approximately 3% of the total seropositive patients being followed in the centers.

In the present study, immunological and virological serum parameters were evaluated to determine those parameters contributing to establish the course of HIV-1 disease progression, fast versus non/slow-progression. Furthermore, among the NP group, the cellular and serum blood factors associated with the progression of HIV-1 disease in those individuals who were initially asymptomatic at enrollment were investigated.

The NP status, as opposed to the FP one, was chiefly correlated to the p24 antibody, and inversely correlated to circulating IFNα titers. Among Non/Slow Progressors (NP), the subsequent progression toward AIDS, as determined by clinical and/or biological symptoms, was chiefly associated with low tat antibody levels and high p24 antigen. Of interest, high antibody levels to tat peptide (AA1-15) but not to other regions of the protein were also correlated to stability (not shown).

The high inverse correlation found between anti-tat antibodies and p24 antigenemia in all subjects gives a unique status to the tat protein compared to the other HIV-1 proteins investigated in this study, namely gp120, nef and p24. Because progression of the disease in asymptomatic HIV-1 infected individuals (NP subjects at enrollment) appears to be very much associated (p=0.006) with low anti-tat antibodies (<0.31 O.D.), anti-tat antibodies are a critical marker of initiation of immune dysfunction (disease progression) in advance of p24 antigenemia, which reflects viral replication activity.

Antibodies to tat recognize tat protein in the extracellular milieu but not within HIV-1 infected cells. Thus, the anti-tat effect of these specific antibodies prevents this viral protein from targeting uninfected cells including immune cells (Viscidi et al, 1989; Chirmule et al, 1995). In this context, tat antibodies could contribute to locally prevent the tat-induced cellular immune suppression in those lymphoid foci with acute infection and extracellular tat, as they do in vitro in activated PBMC infected by HIV-1 (Zagury et al, 1998a). Anti-tat antibodies may neutralize the capacity of tat to inhibit the immune response to recall antigens, to prime non-infected T cells for activation-induced apoptosis, and, further, to prime non-infected cells for infection by HIV-1 (Li et al, 1995). Thus anti-tat antibodies may control in vivo HIV-1 replication indirectly by preventing tat-mediated immune suppression and programmed death of activated immune cells.

It has been previously demonstrated that the CTL response is initially effective in controlling HIV-1 infection, and the loss of this response is associated with progression of HIV-1 infection toward AIDS (Borrow et al, 1997; Goulder et al, 1997). The role of CD8 cells in mediating control of HIV-1 infection may be through direct cytolysis of HIV-1 infected cells and/or by release of soluble factors (i.e., C-C chemokines) within the context of the cellular immune response. CD8⁺ cells from multiply exposed, but non-infected, individuals have been demonstrated to confer protective immunity to hu-PBL-SCID mice challenged with HIV-1 infection (Zhang et al, 1996).

tat-induced loss of cellular immune reaction, particularly the absence of effector CTLs, appears to be accompanied by decrease of anti HIV-1 C-C chemokines (Zagury et al, 1998a). As a consequence, loss of control of virus replication ensues in infected patients (Borrow et al, 1997; Goulder et al, 1997; Zagury 1997). Circulating tat antibodies may exert a protective role by blocking the tat-induced suppressive effect on the cellular immune response to infected cells, as well as other opportunistic pathogens, resulting in the control of viral replication and maintenance of immune functions. The control of viral replication by tat antibodies is highly suggestive by the inverse correlation (p<0.001) found in the GRIV cohort between tat antibodies and p24 antigenemia (Table 3 and FIG. 4A).

These studies showing that tat antibodies correlate with non-progression and previous in vitro results demonstrating the role of extracellular tat in suppressing T cell response to antigen including C-C chemokine production (Zagury et al, 1998a) and CTL activation (Zagury et al, 1997), in promoting activation induced T cell apoptosis and priming non-infected cells for virus replication (Li et al, 1997), prompted the present inventors to establish a new strategy based on anti-tat active immunization (Gringeri et al, submitted). The present study provides a strong rationale to include tat immunogen as an integral AIDS vaccine component in order to permit sustained cellular immune responses in the face of the immunopathologic effects of HIV-1 infection. Toward this end, a strategy for tat preparation, namely tat-Toxoid was developed (Gringeri et al, submitted). This strategy should permit successful application of conventional vaccine strategies using anti-HIV-l CTL-inducing antigens (Picard et al, 1992; Achow et al, 1990; Nixon et al, 1991). tat immunization may be an effective strategy to circumvent tat-induced immune suppression and viral dissemination. In the absence of these effects, the immune system may be capable of responding successfully in response to HIV-1 infection. Thus the tat-Toxoid approach may be an essential component of therapeutic as well as preventive vaccine strategies against AIDS.

### EXAMPLE 2

### Material and Methods

Subjects

Fourteen HIV-1-infected but asymptomatic patients volunteered to participate in this open phase I pilot study and signed the local Ethics Committee-approved informed consent.

Of 14 anti-HIV-positive asymptomatic patients enrolled in the study, 11 were men with hemophilia aged 19 to 39 years (median, 31 years). The remaining 3 patients were women aged 20 to 36 years (median, 33 years) who had been infected by heterosexual contacts. The duration of HIV-1 infection ranged from 4 to 15 years (median, 12 years). Eight of 14 patients had never received antiretroviral treatment, whereas the remaining 6 had been on antiretroviral treatment for at least 1 year. Among these 6 patients, 4 had been treated with three drugs, including proteinase inhibitors, for at least 6 months. Furthermore, 6 of 14 enrolled patients had been previously anti-IFN-a immunized, of whom 3 were receiving antiretroviral treatment.

CD4⁺ cell counts ranged from 219 to 453 cells/mm³ (mean·SD 293·70cells/mm³) at the time of enrollment. Other immunologic and virologic parameters are shown in Table 8. All 11 hemophiles were also infected with hepatitis C virus (HCV).

Immunization

Immunogen

TAT toxoid was prepared by chemical inactivation of recombinant HIV-1 TAT protein purified by solubilization in 6M guanidine and HCl-containing buffer followed by chromatography on nickel agarose (NTA, Quiagen, Hilden, Germany). The protein was expressed in Escherichia coli as a fusion protein in pRSETA (Invitrogen, San Diego, Calif., U.S.A.) and contained six hystidine residues (i.e., a nickel-binding site). HIV-1 TAT cDNA expression vectors were derived from HIV-1-MB, pCV1. Purified TAT protein, but not TAT toxoid, exhibited strong biologic activity as measured by the CAT assay on HeLa cells. The activity of native TAT was inhibited by murine anti-TAT Ab.

HIV-1-TAT toxoid was manufactured by Bio Sidus (Buenos Aires, Argentina) under Good Laboratory Practice conditions.

HIV-1 Tat Immunizing Preparations

Priming preparation: 100 μg of HIV-1 TAT toxoid in water-in-oil emulsion (incomplete Freund adjuvant HFA with Seppic mineral oil, ISA051).

Booster preparation 50 μg of HIV-1 TAT toxoid in water-in-oil emulsion HFA, Seppic ISA051).

Immunization Schedule

HIV-1-seropositive subjects were intramuscularly injected with HIV-1 Tat priming preparation and boosted every 1 to 2 weeks (immunization A) or every 4 weeks (immunization B) until a twofold or greater rise of the preimmunization level of anti-Tat Abs, determined in optical density (OD), was detected by enzyme-linked immunosorbent assay (ELISA) in the sera collected 7 to 14 days after each injection. Subsequently, the anti-Tat immunization was maintained by boosting with Tat every 3 to 4 months according to the anti-TAT Ab levels. Schedule A included 8 patients and schedule B included 6 patients.

Tolerance Evaluation

Safety and tolerance were assessed by clinical examination, patient symptoms, and laboratory evaluation (biochemistry and complete blood cell counts). Local and systemic side effects were recorded 7 days after each injection.

Safety Evaluation

Signs and symptoms related to AIDS progression were carefully looked for over the follow-up period by clinical examination. Any change in antiretrovial treatment was noted.

CD4⁺ cell counts (PACS with Ortho Diagnostics monoclonal antibodies, Raritan, N.J., U.S.A.), HIV-1 plasma viremia (RNA PCR, ELISA kit from Viroquant, Paris, France) and HIV-1-p24 antigenemia (ELISA, NEN-Du Pont, Nemours, France) were the main parameters followed for evaluation of safety. The Viroquant ELISA kit for HIV-1 RNA has a sensitivity threshold of 10 cq copies/mL. The ELISA kit for p24 antigenemia has a sensitivity threshold of 4.4 pg/mL. In addition, liver function tests were accurately monitored in HCV-positive patients.

Immunogenicity Evaluation

Immunogenicity of TAT toxoid was evaluated by assaying anti-HIV-1 TAT Abs by ELISA 7 to 14 days after each injection. The serum anti-Tat Abs were detected by standard ELISA assay using flat-bottomed, 96-well Costar plates (Costar, Cambridge, Mass., U.S.A.). Tat recombinant protein was fixed on the plate at 50 ng/well. Sera at 1:500 dilution were tested according to the standard ELISA procedure, and the results were expressed as OD values. Circulating Tat Ab levels varied among infected patients. In contrast, in all infected but not immunized patients (>100 subjects tested), consecutive serum samples collected within a 1-year period and tested in the same experiment under the same ELISA condition do not exhibit variability in their Tat Ab levels less than 1.5-fold). This is the reason why responders were defined as those patients showing an increase in anti-HIV-1 TAT Ab levels of twofold or more from preimmunization values. Cell-mediated immunity (CMI) and delayed-type hypersensitivity (DTH) response toward HIV-1-TAT protein were evaluated at random by lymphoproliferative response and skin tests, respectively. CMI was assayed by T-cell proliferation measured by ³H-thymidine incorporation; fresh peripheral blood mononuclear cells (PBMCs) from patients were cultured for 6 days in 96 well round-bottomed culture plates in the presence (test samples) or absence (control samples) of 10 μg/mL of Tat toxoid. Eighteen hours before the completion of the culture, 0.5 μCi of thymidine was added to each well. Cells were then harvested, and thymidine incorporation in cell DNA was measured in a β counter and expressed as counts per minute (cpm) per milliliter. Results are expressed as the ratio or cpm in test samples to cpm in control samples, and a ratio of >2 was considered as positive (Picard et al., 1992). Administration of an intradermal injection of Tat toxoid (10 mg) in 100 mL phosphate-buffered saline (PBS) was carried out for the DTH skin test. Positive skin tests corresponded to a papula of >1 cm diameter measured at 48 hours.

Six nonimmunized HIV-1-seropositive and 6 sero-negative subjects matched for age, gender, and CD4⁺ cell counts (the latter only in HIV-seropositive patients) were used as controls for these tests.

### Results

Safety and Tolerance

Seven of 14 patients entered in the study reported mild fever (up to 38□C) lasting 12 to 48 hours after the first injection (priming preparation). Nine of 14 patients complained of pain and swelling at the site of the first injection (priming preparation) requiring anti-inflammatory drugs. Pain and swelling resolved 2 to 7 days after the injection in all cases. No side effects were reported on successive injections.

Clinical evaluation showed no signs of HIV-1 disease progression and no concomitant diseases related or unrelated to HIV-1 infection. Antiretroviral treatment did not have to be changed during immunization and follow-up in any patients.

Absolute CD4⁺ cell counts increased significantly after immunization, with a mean difference from preimmunization values of 60 cells/mm³ (95% confidence interval, 34-85 cells/mm³; Student's t-test for paired samples, t=4.99; P=0.0002), whereas the CD4⁺ cell percentage did not change significantly (Table 7).

Up to the last follow-up visit, HIV-1 plasma viremia did not significantly change after immunization from preimmunization levels, although a trend toward a decrease was observed (mean difference from preimmunization values, −0.63 eq RNA copies×10 mL; 95% confidence interval, −1.341 to +0.067 eq RNA copies×10⁴ mL; Student's t-test for paired samples, t−1.95; P=0.0725); baseline and follow-up levels are shown in Table 7.

HIV-1 p24 antigenemia did not change significantly after immunization from preimmunization levels of Student's

t-test for paired samples, t=1.76, P=0.10), even though 4 of 14 patients showed a decrease (Table 7). Liver function tests as well as other biochemical or hematologic parameters did not show any difference from preimmunization values.

TABLE 7
Absolute CD4⁺ Cell Counts HIV-1 Plasma Viremia, and Antigenemia in 14 HIV-1-Seropositive
Patients Immunized With Inactivated Recombinant HIV-1 TAT Preparation

| | | CD4⁺ Cell Counts/mm³ (%) | | HIV-1 Plasma Viremia^{α} | | HIV-Ag (pg/mL) | |
|---|---|---|---|---|---|---|---|
| | Follow-Up | | At End of | | At End of | | At End of |
| Code | (months) | Preimmunization | Follow-UP | Preimmunization | Follow-UP | Preimmunization | Follow-UP |
| 1 | 8 | 284 (28.4%) | 345 (34.5%) | 5.75 | 5.71 | 11 | 4 |
| 2 | 8 | 453 (16.8%) | 534 (18.4%) | 2.06 | 2.15 | 32 | 3 |
| 3 | 12 | 227 (28.4%) | 345 (34.5%) | 0.35 | <0.001 | 17 | 4 |
| 4 | 3 | 287 (22.1%) | 252 (21.0%) | 0.49 | 0.70 | 3 | 3 |
| 5 | 5 | 282 (28.2%) | 319 (22.8%) | 3.34 | 0.91 | 4 | 4 |
| 6 | 9 | 224 (11.8%) | 294 (12.8%) | 5.23 | 3.90 | 3 | 3 |
| 7 | 3 | 313 (24.1%) | 443 (23.3%) | 5.39 | 5.55 | 4 | 4 |
| 8 | 4 | 275 (30.6%) | 314 (31.4%) | 4.12 | 3.18 | 5 | 4 |
| 9 | 6 | 274 (8.3%) | 386 (9.9%) | 5.08 | 5.74 | 4 | 5 |
| 10 | 8 | 295 (22.7%) | 372 (26.6%) | 4.02 | 3.9 | 5 | 4 |
| 11 | 4 | 219 (16.9%) | 235 (23.5%) | 4.01 | <1 | 8 | 3 |
| 12 | 12 | 272 (20.9%) | 340 (30.9%) | 4.74 | 2.30 | NA | 4 |
| 13 | 9 | 435 (33.5%) | 478 (28.1%) | 4.30 | 4.07 | 3 | 4 |
| 14 | 3 | 257 (13.5%) | 274 (13.7%) | 2.05 | 2.90 | 3 | 3 |

^{α}HIV-1 plasma viremia is measured in equivalent copies 10,000/ml (see methods)
NA—not available

Immunogenicity

Eight patients were assigned to induction schedule type A on a voluntary basis and the remaining 6 patients followed immunization scheme B. All patients responded to TAT immunization with an increase in Abs to Tat ranging from twofold to eightfold (Table 8), including patients who were treated with antiretroviral drugs, immunized against IFN-α or both. Patients received 1 to 8 injections to induce and maintain an Ab response during a follow-up period of 3 to 12 months (median, 8.5 months). An Ab response defined as a twofold or more rise of anti-HIV-1 TAT Ab level from preimmunization levels, was achieved with 1 to 5 injections (median, 3 injections) both in patients with induction type A (injection every 1-2 weeks) or type B (injections every 4 weeks).

Four of 8 tested patients showed a positive DTH response to intradermal injection of inactivated recombinant TAT protein with persistence of skin induration (papula) up to 72 hours (Table 8).

TABLE 8
Immunogenicity of Anti-HIV-1 TaT Toxoid in
HIV-1-Seropositive Patients

| | | | | Anti-TAT | |
|---|---|---|---|---|---|
| | | | | Antibodylevel (OD) | |
| | Immunization | | | DTH | CMI |
| Code | Type (A/B) | Preimmunization | Peak | Response | Response |
| 1 | B | 0.240 | 1.230 | NA | NA |
| 2 | A | 0.104 | 1.526 | NA | Positive |
| 3 | B | 0.217 | 0.537 | Negative | Negative |
| 4 | B | 0.214 | 1.063 | Positive | NA |
| 5 | A | 0.446 | 1.612 | NA | NA |
| 6 | A | 0.213 | 0.664 | Negative | Negative |
| 7 | A | 0.204 | 1.139 | NA | NA |
| 8 | B | 0.104 | 1.045 | Positive | Positive |
| 9 | A | 0.422 | 0.812 | Negative | NA |
| 10 | B | 0.125 | 1.492 | NA | NA |
| 11 | A | 0.252 | 0.682 | Negative | NA |
| 12 | A | 0.410 | 2.219 | Positive | Positive |
| 13 | A | 0.162 | 0.508 | Positive | Positive |
| 14 | B | 0.120 | 2.012 | NA | NA |

DTH delayed-type hypersensitivity:
GMI, cell-mediated immunity:
NA, not assayed:
OD, optical density.

Four of 6 patients showed a consistent increase of proliferative response to the Tat antigen in assays of CMI (Table 8) that was twofold to 10-fold compared with cells cultured without any antigens. In 3 positive patients (P3, P8, and P12), frozen PBMCs collected prior to Tat immunization were available. T-cell proliferation of these cells following Tat toxoid stimulation did not yield any positive proliferative response (data not shown). Thus, Table 8 shows the use of ELISA anti-Tat to monitor the patients and show that the titer of the patients for anti-Tat antibodies has increased following immunization. This demonstrates that the measure of anti-Tat antibodies is a good test to follow-up patients and provide a prognosis of disease evolution.

None of the HIV-1-seropositive or seronegative control subjects exhibited an increase of anti-TAT Ab levels and CMI response to Tat protein. Non of the controls showed a positive response to DTH skin test to Tat protein.

### Discussion

This pilot phase I study was designed to demonstrate safety, tolerance, and immunogencity of a preparation of HIV-1 TAT toxoid in immunocompromised HIV-1-seropositive patients, even those concurrently treated with antiretroviral drugs, immunized against IFN-α or both.

This study showed that anti-HIV-1 TAT immunization in these patients was safe and well tolerated. Biochemical, hematologic, immunologic, and virologic parameters as well as clinical evaluation did not suggest any toxic effect of immunization. Moreover, absolute CD4⁺ cell counts appeared to increase after immunization in most of patients, and in some patients, a decrease in HIV-1 plasma viremia and p24 antigenemia occurred. Additionally, none of the immunized patients showed an increase in virologic parameters during the follow-up period, even though many were not given antiretroviral drugs. These results are consistent with the protective role of anti-TAT Abs, (Lolli et al., 1995; Krone et al., 1988; and Reiss et al., 1991) correlated inversely with p24 antigenemia and illustrated in nonprogressor patients (Zagury et al., 1998).

This study showed that the immunizing preparation of HIV-1 TAT toxoid was highly immunogenic; all patients who were at different degrees of HIV-1-related immunodeficiency responded and some did so after just one injection. Immunization induced CMI, a positive cell-mediated immunity, and even a DTH response in at least some of the patients evaluated.

It should be stressed that this active immunization aiming at reducing the TAT toxin pathogenic effects (Viscidi et al., 1989; Chirmule et al., 1995 and Zagury et al., 1996), can be used in combination with antiretroviral treatments, anti-IFN-α immunization, or both. It will be important to expand the observations derived from this pilot phase I study in a larger group of patients.

Because extracellular TAT can be considered an HIV-1 strategy to induce immune suppression and apoptosis on nonifected immune cells (Viscidi et al., 1989; Chirmule et al., 1995 and Zagury et al., 1996), we propose to counteract these deleterious effects by an anti-Tat immunization. Tat toxoid could be an integral vaccine component for seronegative (preventive) or seropositive (therapeutic) people. Such a vaccine preparation comprising HIV-1 CTL-inducing epitopes should combat cellular immune suppression, (Zagury et al., 1998) viral replication (Zagury et al., 1998) and natural progression toward AIDS, (Goulder et al., 1996; Borrow et al., 1997 and Zagury, 1997) if not viral entry.

### EXAMPLE 3

### Methods and Materials and Methods

The open, controlled, phase I vaccine trial was designed to evaluate safety and immunogencity of a Tat toxoid preparation (Gringeri et al., 1998) in 5 seronegative study subjects, over a 6-month follow-up period.

Immunizing Reagents

The immunogen referred to as Tat toxoid-was a chemically inactivated recombinant HIV-1 Tat protein (Le Buanee et al., 1998; and Gringeri et al., 1998) adjuvanted with incomplete Freund adjuvant (IFA), which consisted of the mineral of ISA 051 from Seppic (Paris, France). TAT toxoid was prepared by chemical inactivation of recombinant HIV-1 TAT protein purified by solubilization in 6 M guanidine. HCl-containing buffer, followed by chromatography on nickel agarose (NTA, Qiagen, Hilden, Germany). The protein was expressed in Escherichia coli as a fusion protein in pRSETA (Invitrogen, San Diego, Calif., U.S.A.) that contained six hystrix residues (nickel binding site). HIV-1 TAT cDNA expression vectors were derived from HIV-1 pCV1. Purified TAT protein but not TAT toxoid exhibited a strong biologic activity as measured by the CAT assay on HeLa cells. The activity of native TAT was inhibited by murine anti-TAT antibodies. Inactivation of Tat toxoid was evaluated by absence of reactivity tested by CAT assay (Le Buanee et al.; Gringeri et al., 1998).

Immunization Protocol

Tat toxoid (70 μg) in phosphate-buffered saline (PBS, 0.4 mL) emulsified with IFA (0.4 mL), was injected intramuscularly from 1 to 3 times at 1-month intervals, to ascertain immunogencity with different vaccine schemes and safety after multiple injections.

Serum and peripheral blood mononuclear cells (PBMCs) were separated from blood collected prior to the first injection (control) and 8 days after each injection.

Study Subjects

Five healthy volunteers (3 men, 2 women), between 25 and 46 years of age, were enrolled (A-B, Table 9). HIV-1 antibody negativity was tested by enzymed-linked immunosorbent assay (ELISA) with confirmatory Western blot test. Inclusion criterian were absence of active or chronic diseases, age between 18 and 65 years reliability to adopt preventive measures to prevent HIV-1 infection no exposure to risk of HIV-1 infection, in the previous 3 months, no active signs or symptoms of any acute disease, and signed informed consent. Two more study subjects with the same characteristics (1 man, 1 woman) were enrolled as controls (F and G, Table 9).

One subject received a single injection of TAT toxoid vaccine intramuscularly. These study subjects were primed with two injections of the same preparation and the remaining study subject received TAT toxoid three times.

Any signs and symptoms were carefully investigated by clinical examination and interviews over the first 7 days after each injection and monthly thereafter for the entire 6-month follow-up period. Complete blood cell counts, T-cell phenotype, and renal and liver function tests were carried out before and after immunization.

Immunogencity

The humoral response to Tat toxoid relied on the determination by ELISA of circulating anti-Tat antibody levels on sera collected between 7 and 14 days after each injection and monthly thereafter using biologically active recombinant Tat as the detecting antigen.

The serum anti-Tat antibodies were detected by standard ELISA assay using Costar (Cambridge, Mass., U.S.A.) plates (FB, 96 wells 3590). Tat recombinant protein was fixed on the plate at 50 ng/well. Sera at 1:500 dilution were tested according in the standard ELISA procedure and the results were expressed as optical density (OD) values. Study protocol defined as responders those study subjects showing an increase of anti-HIV-1 TAT antibody levels of twofold or more from preimmunization values. This cutoff is based on the observation of HIV-1-infected but not immunized patients (>100 study subjects tested), who did not exhibit variability in their Tat antibody levels (less than 1.5-fold) in consecutive serum samples collected over a 1-year period and tested in a same experiment (under the same ELISA measurements). Anti-Tat antibodies titration was done 4 to 6 months after the first injection and expressed as the highest dilution giving a positive reaction measurable by ELISA.

Assessment of the cellular response relied on both delayed-type hypersensitivity (DTH) response toward HIV-1 TAT protein and on in vitro cell-mediated Immunity (CMI) by T-cell proliferation of Tat toxoid-stimulated PBMCs measured by ³H thymidine incorporation test. Fresh PBMCs from study subjects were cultured for 6 days in 96-well round-bottom culture plates in the presence (test samples) or absence (control samples) of 10 μg/ml of Tat toxid. At 18 hours before completion of the culture, 0.5 μCi of thymidine was added to each well. Cells were then harvested and thymidine incorporated in cell DNA was measured in a β-counter and expressed as counts-per-minute per milliliter (cpm/ml). Results were expressed as proliferation index (PI). PI=cpm in test samples: cmp in control samples. A PI>1 was considered positive (Picard et al., 1990).

Administration of intradermal injection of Tat toxoid (10 μg) in 100 μl PBS was carried out for the DTH skin test before anti-Tat vaccination and after immunization. Positive skin test results corresponded in a papula of >0.5 cm in diameter measured after 48 to 72 hours.

### Results

Safety and Tolerance

Administration of the Tat toxoid preparation one to three times did not result in any untoward local or systemic reactions and was well tolerated by all the subjects. Even the study subject who received three intramuscular injections of the water-in-oil emulsion did not complain about local pain or any other discomfort. No change in complete blood cell counts and T-cell phenotypes, or in renal and liver fuction tests was observed in any of the patients.

Humoral Response

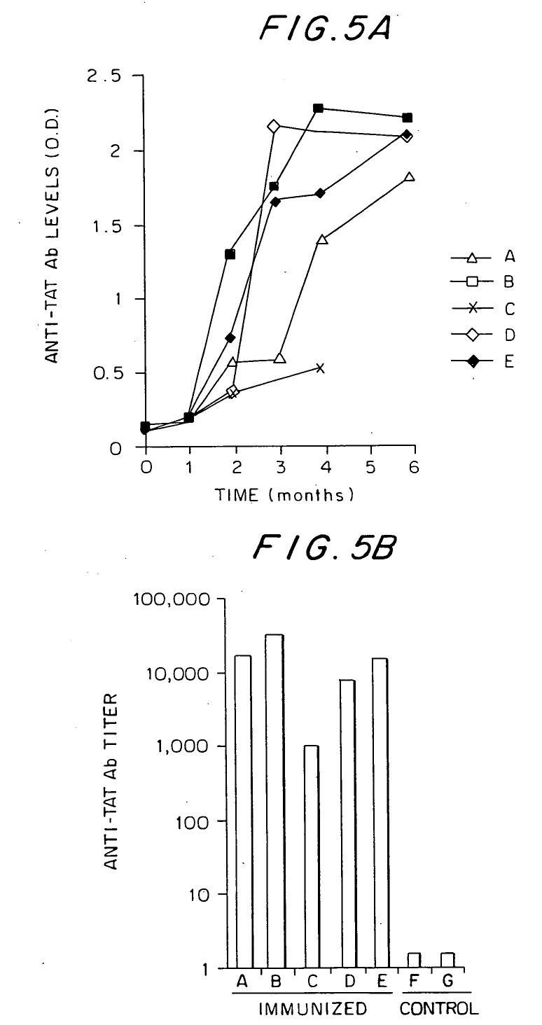

The 5 immunized study subjects exhibited high anti-Tat antibody levels in their serum following the first, second, and/or third injection (FIGS. 5A and 5B) with an increase of antibody levels ranging from threefold to more than 10-fold from preimmunization values. No detectable anti-Tat antibody levels were shown by ELISA in control study subjects (<0.250 OD). After 4 to 6 months from the first immunizing injections, 5 of 5 immunized study subjects exhibited high titers of anti-Tat antibodies ranging from 1:1,000 to 1:64,000 (FIG. 5B, Table 9), whereas no dectable titers were found in controls (<1:250). The subjects exhibiting the highest level of anti-Tat antibodies should be those that will have better protection against HIV-1 infection.

Cellular Response

The 4 tested study sujects showed a positive DTH response to Tat characterized by a well-formed 1- to 3-cm red papula occurring at 48 hours following intradermmal injection of Tat toxoid and persisting at 72 hours (Table 9), whereas no skin reaction was observed in control study subjects.

Cell-mediated immunity, as measured by T-cell proliferation of PBMCs following Tat toxoid stimulation was also markedly increased in the 5 immunized individuals but not in nonimmunized voluteers (Table 9). Proliferation index varied from 6 to 63 in vaccinees, whereas it was <1.5 in controls.

TABLE 9
Immune response of seronegative study subjects to Tat toxoid

| | | | CD4 cell | | | | | |
|---|---|---|---|---|---|---|---|---|
| | | | count (%)^{a} | | Tat toxoid | | | |
| Subjects | Age (y) | Gender | T0 | T1 | Injections | DTH^{b} | Anti-Tat Ab titer^{c} | CMI^{d} |
| A | 46 | F | 40.9 | 44.9 | 2 | + | 16,000 | 63 |
| B | 43 | M | 50.8 | 52.1 | 1 | + | 32,000 | 11 |
| C | 25 | F | 49.0 | 44.1 | 2 | NA | 1,000 | 6 |
| D | 29 | M | 37.0 | 31.9 | 3 | + | 8,000 | 32 |
| E | 35 | M | NA | NA | 2 | + | 16,000 | 10 |
| F | 30 | M | 48.5 | 43.6 | 0 | − | <250 | 1.2 |
| G | 32 | F | 43.8 | 42.2 | 0 | − | <250 | 1.0 |

^{a}T0, preimmunization values: T1, values 4 to 6 months after the first immunizing injection.
^{b}Delayed-type hypersensitivity test was performed by Intradermal injection of Tat toxoid (0.5 μg in 100 μl).
^{c}Anti-Tat Ab titers were tested on serum samples collected 4-6 months after the first injection.
^{d}Cell-mediated immunity was assessed by T-cell proliferation measured by ³H thymidine incorporation. Results are given as proliferation index (PI). PI = cpm (dxp): cpm (control).
NA not available:
DTH, delayed-type hypersensitivity:
CMI, cell-mediated immunity:
Ab, antibody:
cpm, counts per minute.

### Discussion

This open, controlled study was designed to evaluate safety and immunogenicity of an active immuniztion against HIV-1 Tat protein using an inactiviated recombinant Tat toxoid in healthy HIV-1-seronegative study subjects. An inactivated but immunogenic Tat (i.e., Tat toxiod) was used as immunogen to avoid damages induced by native Tat on various tissues including the CNS (Shi et al., 1998) and the immune system (Viscidi et al., 1989), to which introduction of Tat toxin might introduce hazards particularly in those study subjects with an autoimmune predisposition or those whose immune systems were compromised by chronic infections, chronic parasitic infestation, or as a result of malnutrition. Immunization with Tat toxoid, in a water-in-oil emulsion, was safe and well-tolerated by all seronegative study subjects.

Anti-Tat immunization induced high levels of circulating anti-Tat antibodies in all immunized study subjects; this antibody response was achieved even after one immunizing injection and was accompanied by a cellular resonse, as shown by T-cell proliferation in vitro and by skin test in vivo. Furthermore, individuals A, B, and E, who were first immunized 1 year previously, continued to maintain a high titer of circulating Tat toxin antibodies (16,000⁻¹, 8,000⁻¹ and 16,000⁻¹, respectively). Of interest in individual D, who was immunized at the same time with Nef toxoid and p24 protein, the titers of circulating antibodies and the cellular response directed against these HIV-1 antigens increased greatly (data not shown), as well as those directed against Tat (Table 9).

In conclusion, the presence of high levels of circulating anti-Tat antibodies should antiagonize Tat toxin released into the extracellular compartment by infected cells and should thus prevent the Tat-induced immunosuppression of uninfected T cells. Thus, following HIV-1 exposure of uninfected individuals immunized with Tat toxoid and other CMI-inducing HIV-1 antigens, such as Env, Gag or Nef peptides, the presence of high levels of circulating antibodies against Tat should control the immunosuppressive effect of Tat toxin released during acute infection and allow the cellular response induced by HIV-1 structural antigens to develop. The almost immediate release of high level of β-chemokines could contribute to enhancement of the host resistance to HIV-1 infection (Zagury et al., 1998). A phase II/III trial will be conducted to confirm these assertions.

### EXAMPLE 4

With the high success rates seen with HAART today, STI likely represents a powerful tool to quickly evaluate the clinical benefit of immune therapeutic approaches including vaccines. In this study, the present inventors took advantage of a 2 year STI study initiated at Hospital St Pierre, Brussels, which followed the Sanofi-Pasteur Tat Toxoid vaccine trial in which vaccinees interrupting HAART were followed by their physicians, who were blinded to their vaccination status and their Tat immune responses, under EU ARV treatment recommendations while undergoing STI (FIG. 6). Two major findings emerged from confrontation of clinical outcome of patients with follow-up of their Ab reaction to Tat (Tables IIa and IIb).

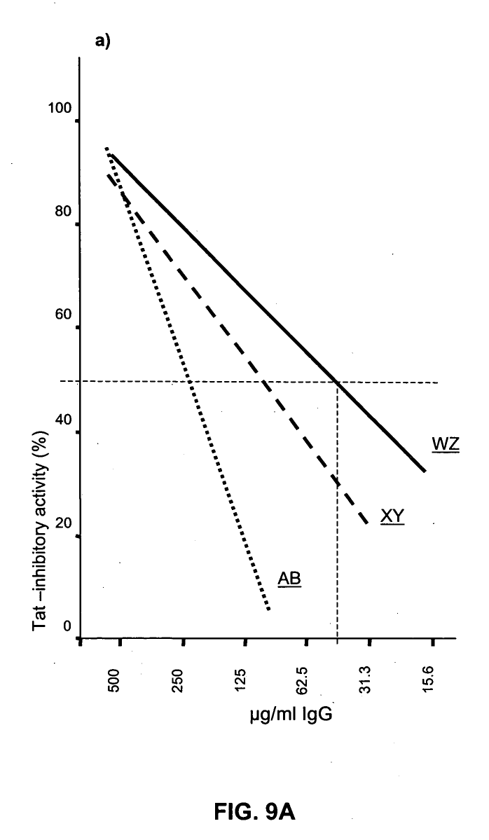

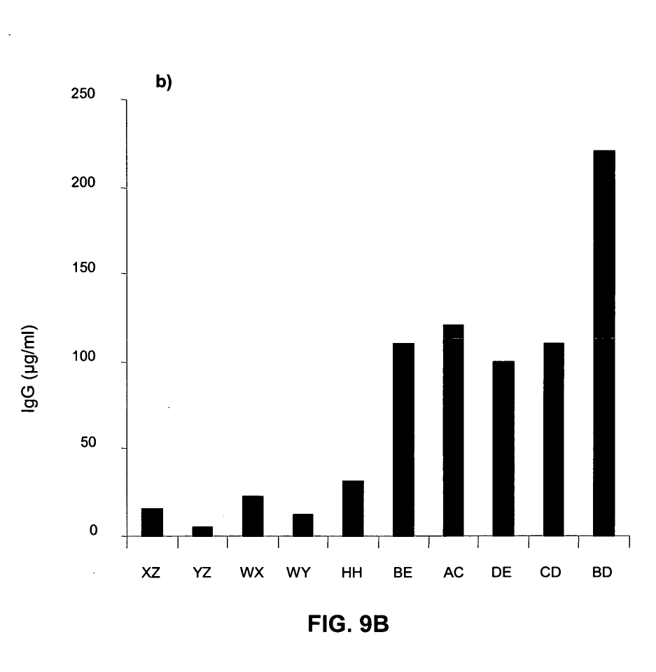

1) Tat Toxoid-induced development of positive Tat Ab titers was followed by prolonged delay of HAART resumption. This result was statistically significant for the total population under STI, and particularly for the subgroup of volunteers with established chronic infection. Remarlably, all 6 Ab responders receiving DcChol adjuvanted Tat Toxoid were sustained without HAART for the duration of the STI follow-up. Induction of positive serum ETIC (score>2) in Tat Toxoid immunized patients was also associated with prolonged absence of HAART. For the volunteers receiving Tat Toxoid, antibody titer and serum ETIC strongly correlated and may, in fact, represent 2 expressions of the anti-Tat antibody response triggered by this immunogen. This is based on the in vitro tests showing that high avidity Abs purified from antibody responders sera strongly inhibited extracellular Tat activity (FIGS. 9A and 9B). Noteworthy the use of other Tat immunogenes including native Tat (Cafaro et al., 1999) or tat DNA (Liang et al., 2005) did not trigger any ETIC which could account for reported absence of correlation between antibody response and efficacy in these experiments (Table Ia).

2) Absence of serum ETIC preceded or was concomitant with HAART resumption. Loss of ETIC i.e. <20% reference serum value may occur at a time immediately preceding HAART prescription, as in volunteers AB or DE but, most often, sometime before as in volunteers BC, BE. The latter finding may account for 1) why volunteers, though few (n=5) with absence of serum ETIC at the end of STI, still remained off HAART and 2) why volunteers still exhibiting an ETIC at terminal stage of STI did not resume HAART.

These data are of interest for AIDS pathogenesis should further trigger medical applications both at diagnosis and therapeutic levels.

Clinical evolution toward AIDS is dependent on multiple interacting viral and host factors, such as the viral load and CD4 cell count. Both are used as major predictive markers of prognosis and indicators for treatment initiation. Scientists, however, disagree as to the interaction between these 2 factors, some arguing that CD4 T cell decline is basically due to direct killing of infected cells by HIV (Ho et al., 1995; and Wei et al., 1995), while others claim that the loss of infected cells is due to HIV-induced immunosuppression of uninfected cells (Finkel et al., 1995). The latter idea is documented by experiments showing the dual pathogenic role of extracellular Tat within infected lymphoid foci acting on both infected and bystander uninfected CD4 T-cells (Nardelli et al., 1995; Westendorp et al., 1995; and McCloskey et al., 1997). The results of the present inventors support this hypothesis.

Since both positive serum ETIC was associated with prolonged absence of ARV treatment and absence of serum ETIC with needs of HAART, the present inventors consider serum ETIC as a relevant prognosis factor to predict clinical outcome and help monitoring chemotherapy.

The prolonged absence of HAART in patients with positive anti-Tat Ab response particularly those who had received Tat Toxoid with DcChol adjuvant (50%) underscores the therapeutic value of this vaccine. Three considerations however should be stressed. First, this vaccine preparation containing an oily adjuvant is chiefly aimed at eliciting neutralizing Abs in order to counteract the deleterious effects of extracellular Tat (Gallo, 2005) but not at triggering an anti-Tat CMI to lyse infected cells (Cafaro et al., 1999; and Liang et al., 2005). Second, since no notable differences in immune status were found between antibody responders and non-responders, it was hypothesized that the weaker reaction to DcChol adjuvanted Tat Toxoid of non-responders from this group may be due to their different individual phenotypic background (Zagury et al., 1998). Furthermore since in Ab responders anti-Tat Ab decrement within 2 years was the rule, following vaccination, boostering injections should maintain high Ab levels. Consequently, antibody production may be improved with a more robust immunization regimen including boostering, as achieved by A. Gringeri, who obtained a persistent positive antibody response in most of vaccinees after 6 monthly administered IM injections of Tat toxoid emulsified in Salk adjuvant followed by biyearly booster injections (unpublished data). Third, like other HIV-1 proteins, Tat has regions of hyper-variability. Consequently Abs induced by Tat Toxoid (strain IIIB clade B) may not be as efficacious, when given to patients infected with HIV-1 clades far distant from the B clade (Butto et al., 2003).

In conclusion, this 2 year STI study supports the concept of a pathogenic role of extracellular Tat protein and further indicates that high avidity Abs inhibiting the effects of extracellular Tat, as those triggered by DcChol-adjuvanted Tat toxoid help in the control of HIV-1 infection at levels which contribute to a prolonged HAART-free status. A large phase 2 multicentered double blind placebo trial using DcChol adjuvanted Tat Toxoid is currently in progress to expand this study.

### Materials and Methods

Pre-STI immunization:

Thirty-two HIV-1 infected patients were enrolled in a double blind randomized placebo controlled phase I/II 24-week clinical trial to evaluate safety and immunogenicity of an HIV-1 recombinant Tat toxoid vaccine with or without adjuvant in collaboration with Sanofi-Pasteur in HIV-1 infected individuals on a stable regimen of HAART for more than 3 months with undetectable viral load and CD4⁺ cells above 350/mm³ at entry. No nadir value of CD4 below 200/mm³was permitted. Enrolled patients were randomly assigned to the 3 groups of vaccinees who received DcChol-adjuvanted Tat Toxoid as the experimental vaccine (group 1), DcChol adjuvant as placebo (group 2) and adjuvant-free Tat Toxoid as a control to assess safety of the immunogen (group 3). Tat Toxoid, an inactivated but immunogenic Tat derivative, was chemically prepared by carboxamidation of recombinant Tat (clone IIB2) and administered at 100 μg per dose in groups 1 and 3. Patients who were at an early stage of infection (primary patients, n=12) started HAART at enrollment while those infected at a later stage already under HAART for years (chronically infected patients, n=20) pursued their treatment. Primary and chronically infected patients were equally distributed in the 3 groups and there was no influence of sex, ethnicity, age, plasma HIV-1 load and immune status including pre-HAART CD4 T cell count between the 3 groups. Patients remained under HAART during the immunizing regimen which comprised 5 doses of vaccine administered intramuscularly at week 0, 2, 4, 8 and 12 (FIG. 1) (Hermans et al., 2003). The trial showed that the Tat Toxoid vaccine preparations were safe given that no vaccinees exhibited any regional or systemic side effects following immunization. Patients enrolled in the Structured treatment interruption (STI):

From the cohort of 32 patients followed in the Sanofi-Pasteur clinical trial (Hermans et al., 2003), 31 patients were subsequently enrolled in the prospective structured treatment interruption trial (STI) locally designed and approved by the IRB (FIG. 6). Four patients GH, OO, PP and WY dropped the study at week 37, 48, 56, and 78, respectively, for personal reasons unrelated to their medical condition. All patients remained with undetectable plasma virus (<50 copies per ml) and CD4⁺ cells above 350/mm³ and gave written informed consent before stopping HAART. Treatment was interrupted in these patients at the end of the Sanofi-Pasteur trial, i.e., 3 months after the last administered dose of vaccine comprised of DcChol adjuvanted Tat Toxoid (experimental group1 n=12, CD4 range: 412-1344/mm³), DcChol (Placebo, group2 n=8, CD4 range: 381-1544/mm³) or Tat Toxoid in water (control for safety of immunogen, group3 n=11, CD4 range: 543-1544/mm³). The STI enrollment period lasted from April 2002 to July 2004 with a 24-month follow-up for each individual. This pilot study aimed at exploring whether the clinical outcome, especially the need for reinitiating HAART was influenced by the individual immune response to Tat Toxin, particularly the one triggered by the DcChol-adjuvanted Tat Toxoid vaccine selected for the sanofi-pasteur PhaseII trial.

Following the STI, a retrospective analysis of CD4 cell count and Ab response to different HIV-1 antigen at the vaccine trial enrolment (time t₀) performed on the subgroup of anti-Tat Ab responders (AbR+) (n=9) compared to the other patients under STI (Ab non R) (n=22) assessed the equal distribution of patients in reference to their immune status. CD4 cell count varied widely among individual of both groups ranging from 1436 to 383 per mm³. Among the Ab responders mean CD4 cell count was 417 and 639 at the time of HAART initiation and just prior starting the Tat vaccine, as compared with a median count of 403 and 755 respectively in the non responder group. Serum Ab titers to different HIV-1 antigens also varied widely among individuals of both subgroups, but no notable difference could be demonstrated between the 2 subgroups in the number of positive response (2 cut off) to each tested antigen. Positive Ab titers a) to gp120 were found in sera from 5 of 9 AbR⁺ and 10 of 21 Ab non R (Fischer's exact 1); b) to gag in sera from 4 of 9 AbR+ versus 8 of 22 Ab non R (Fisher's exact 0.7); c) to Nef in sera from 6 of 9 AbR+ versus 10 of 22 Ab non R (Fisher's exact 0.43); d) to Tat in sera from 6 of 9 AbR⁺ versus 11 of 22 Ab non R (Fisher exact 0.46). Finally inhibitory Tat activity (ETIC) (see below) from sera collected at time to was found over or equal to serum reference in 6 of 9 AbR⁺ and 12 of 22 Ab non R (Fisher's exact 0.69).

Clinical Follow-Up:

All the patients were prospectively followed by their treating physicians at Hospital St Pierre (Brussels). Together with physical examination, CD4 cell count and viral load were checked on a monthly basis during the 1^{st} year, then every 2 months thereafter. Given that all the patients at the onset of the STI had a plasma viral load<50 copies/ml and a CD4 T-cell count greater than 350/μl, criteria for restarting HAART were based on the E.U. guidelines: CD4<350/μl or viral load>100,000 on 2 consecutive samples one month apart or the occurrence of HIV-1 related clinical disease. Of note, since both the patient and their physician were not aware of the previous assignation in the Tat vaccine trial, this STI study was a double blind. In addition, the final results of the analyses of their immune responses to Tat vaccination were available on September 2004, after the end of the 24-month study period of the STI trial, with no influence on the clinical management of these patients.

Serological Assays

Patients sera were collected at different times of the 2 year STI study starting at day 1, which corresponds to the final time (t_{F}) of the sanofi-pasteur Trial, i.e., t_{F}=day 1.

ELISA: Serum anti Tat Ab titer was measured by ELISA using a coating of 1 μg/ml and serum dilutions from 5.10⁻²−10⁻⁶. Results were expressed in μg Ig equivalent of anti-Tat Ab. The cut off of positive responses (≧10 μg/ml) corresponded to 5 fold the titer of the reference serum (2 μg/ml) and background levels (Bg) to titers below reference serum (<2 μg/ml).

Galactosidase/Cat Assay for Tat activity:

The effect on Tat transcriptional activity of patients sera collected at different times of the STI study was measured by analyses of β-galactosidase expression mediated by LTR-beta-gal transfected TZM-B1 cell line (Derdeyn et al., 2000) triggered by the Tat (40-80 ng/ml) protein preincubated with the tested serum and compared to a reference serum unit. In some initial experiments Tat activity was measured by the CAT (Chloramphenicol acetyl transferase) assay using LTR-CAT transfected HeLa-3T1 (Felber et al., 1988) in place of the TZM-B1 cells. In vitro assays for Tat activity were dependent on a series of factors. These included: number of passages of the HeLa cells, the cell culture conditions (culture plates, medium and time of culture), the Tat dose variation (30-80 ng/ml) and whether the tested serum was heated (56° C. for 30 min) or non-heated, the number of freeze-thawings, the dilution and the incubation time of Tat with serum. By preincubating the Tat protein with the test serum the assay allowed the semi-quantitative evaluation of both inhibitory and enhancing Tat transactivation activity triggered by some sera. Given the variability of β-galactosidase/CAT expression occurring in assays carried out at different times, only data from a same experiment could be safely compared. Also in order to integrate the data from different assays, the same control sample was used throughout the study. This reference control was a pool of sera collected from 23 asymptomatic patients, under HAART. Consequently, results from different tests performed under variable conditions could be compared. To evaluate the serum Tat inhibitory capacity (ETIC) inhibiting Tat transcriptional activity, Tat protein (30-80 ng/ml) in solution in HL-1 medium containing β-mercaptoethanol (5×10 ⁻⁵ M), was preincubated for 40 min. at room temperature with tested or reference sera at various dilutions (1:30-80) prior to its introduction in the tested cell culture. Following a 2 hour incubation at 37° C., medium containing Tat protein was replaced by fresh RPMI-FCS 10% medium. After 48 hour culture, cells were lysed, and CAT/galactosidase expression quantified as described (Derdeyn et al., 2000). Serum Tat inhibitory capacity (ETIC) was semi-quantitatively measured as follows:

$$ETIC = \frac{β gal in reference serum - β gal in test serum}{β gal in reference serum} \times 100$$

βgal from cell culture was expressed in pg per mg of protein.

Given the variability of galactosidase and CAT expression observed between various assays, results from each serum sample tested at different times were computed into scores and expressed as score values. Compared to the reference serum unit, ETIC scores were as follows: 100%−80%=4; 79%−60%=3; 59%−40%=2; 39%−20%=1 and <20=background (Bg), the cut off of 2 selected for positivity corresponded to a ETIC index of 50%±10>reference serum value.

To ascertain that serum ETIC was related to Abs which neutralized Tat activity, the galactosidase assay was also performed using Tat protein preincubated with serial concentrations of IgG purified from patients and pool control sera, after passage through a protein G column (Akerstrom et al., 1986).

Avidity of Anti-Tat Abs:

The Biacore 3000 Technology (Biacore AB, France) was used for polyclonal Ab avidity evaluation. After Tat toxoid specific surface preparation from Biacore CM5 Sensor Chip with Biacore Amine Coupling chemistry, active concentration measurements from patients sera purified polyclonal antibodies were first performed and the respective concentrations were expressed in Equivalent monoclonal IgG unit. Kinetics assays were performed on patients and observed affinity constants calculated thru the BiaEvaluation v4.0.1 software, as empirical quantitative parameters that make polyclonal binding patterns possible to compare in terms of avidity. The respective observed affinity constants named KD* were used for comparison.

Statistical Analysis

The Fisher's Exact test was used to evaluate associations between parameters and clinical outcome. Kaplan-Meier survival analysis was performed for the time of restarting HAART.

The Kaplan-Meier survival curves were compared between groups of responders and non-responders using the Log-Rank test. A p value<0.05 for a 2-sided test was considered statistically significant.

### Results

Thirty-one asymptomatic volunteers with undetectable plasma viral load and CD4 count over 350 per mm³ underwent a structured treatment interruption, discontinuing their individual ARV regimen after the completion of the therapeutic Tat toxoid vaccine trial, i.e., 3 months after the last vaccine injection. Patients CH, OO, PP and WY dropped out at week 37, 48, 56 and 78 respectively for personal non medical reasons (Table II). Clinical follow-up included monitoring of clinical symptoms, CD4 T cell numbers and plasma viremia. During the STI, viral rebound occurred in all volunteers within 8 weeks. ARV treatment was not restarted in 16 of 31 volunteers but was prescribed in 15 patients by the treating physicians on the basis of clinical symptoms as well as changes in CD4 count and viral load according to EU regulations (Table II). The distribution of patients remaining HAART-free or resuming HAART (HAART+patients) was not associated with a particular group of volunteers. However, the ability to detect a positive anti-Tat Ab response defined as >10 μg/ml of Tat antibodies which corresponded to 5 fold over the titer of reference serum (see Materials and Methods section) did correlate significantly with clinical outcome in terms of the clinical need for resumption of ARV treatment.

Absence of Treatment with HAART Correlated with Tat Antibody Levels>10 μg/ml.

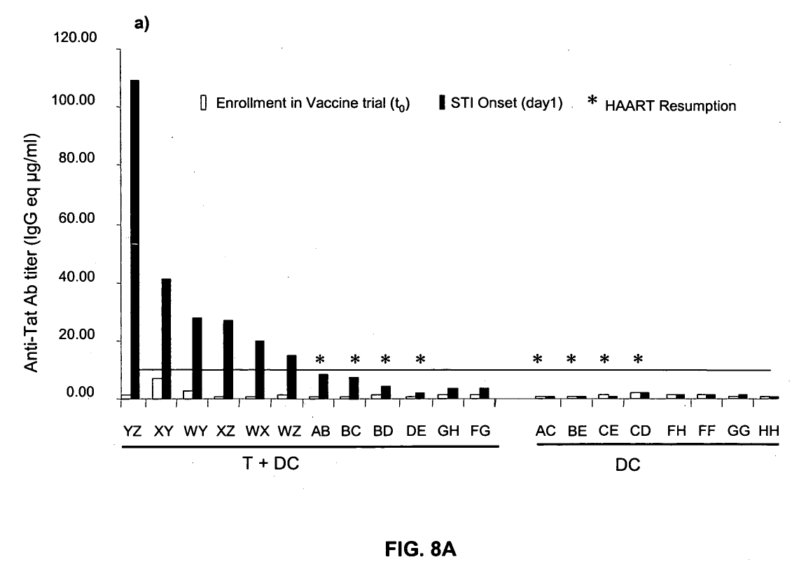

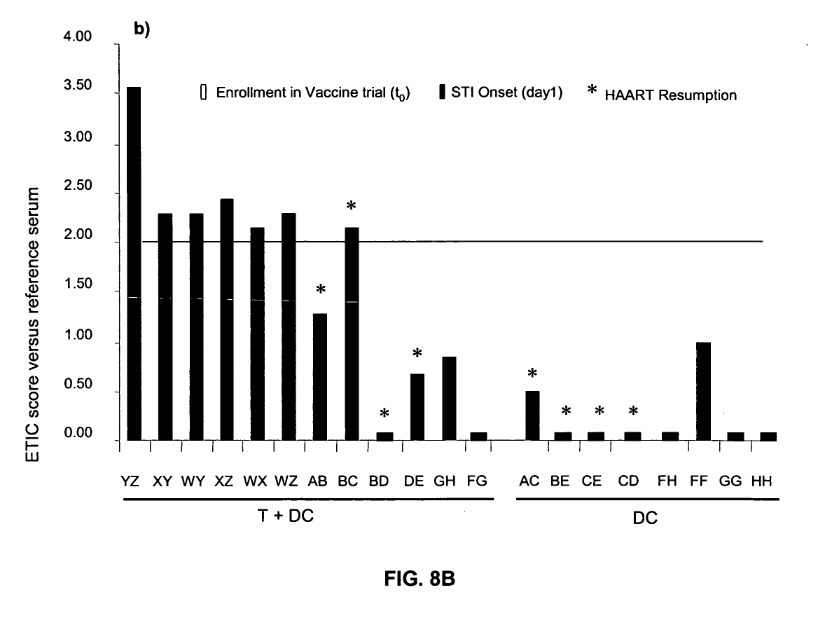

Nine of 23 volunteers previously vaccinated with Tat toxoid vaccine demonstrated anti-Tat antibody levels>10 μg/ml at the time of ARV treatment interruption: none of the 8 volunteers who received adjuvant only exhibited positive Tat antibody titers (Table IIb). Six of 12 volunteers (50%) previously vaccinated with Tat toxoid adjuvanted with DcChol and 3 of 11 volunteers (27%) previously immunized with Tat toxoid alone exhibited serum anti-Tat Ab titers over the cut off for a positive response (Tables IIa and IIc; FIG. 8A). In contrast to those of the DcChol adjuvanted Tat Toxoid group, none of the volunteers in the unadjuvanted group developed significant cell mediated immunity (CMI), as tested in vitro by T-cell proliferation of Tat-activated PBMCs, anticipating a short lasting B cell response following immunization in absence of adjuvant-dependent activation of memory T cells (Gray et al., 1988).

TABLE IIa
Group 1 (patients with DcChol-adjuvanted Tat Toxoid)

| | Clinical evolution of HIV-1 infection during STI | | | | | | | |
|---|---|---|---|---|---|---|---|---|
| | | | | | | | Plasma viremia | |
| | | | | | | | (Copies/ml) | |
| | | | | | | | Samples | |
| | | | | CD4 cells/μl | | | Prior to | |
| | | HIV-1 related | HAART | Samples | | | vaccine | |
| | Stage of | symptoms | resumption | Prior to vaccine | | STI | (Pre | STI |
| Patient | infection² | (week) | (week) | Pre HAART | Time t₀^{3a} | Terminal^{3b} | HAART) | Terminal |
| YZ | Chronic | None | No | 350 | 524 | 336 | 1,350 | 600 |
| XZ | Primary | None | No | 290 | 383 | 311 | 130,000 | 25,000 |
| WY¹ | Chronic | None | No | 700 | 833 | 1041 | >750,000 | 82,000 |
| WX | Chronic | None | No | 500 | 920 | 752 | 14,400 | 36,700 |
| XY | Chronic | None | No | 300 | 489 | 357/297 | 25,000 | 7,200 |
| WZ | Primary | None | No | 800 | 1034 | 580 | 93,000 | 48,000 |
| FG | Primary | None | No | 700 | 575 | 1399 | 100,000 | 47,000 |
| GH¹ | Primary | Orchitis (w 6) | No | 1,000 | 1,436 | 707 | 12,500 | 53,800 |
| | | | | | | (w 37) | | |
| AB | Chronic | Mononucleosis | W 8 | 300 | 759 | 425 | 930,000 | 750,000 |
| | | syndrome (w 8) | | | | | | |
| BD | Chronic | Icteric syndrome | W 24 | 287 | 540 | 306 | 24,000 | 99,000 |
| | | with monoarthritis | | | | | | |
| | | (w 12) | | | | | | |
| DE | Chronic | Acute malaria crisis | W 32 | 260 | 700 | 331 | 10,000 | 196,000 |
| | | (w 28) | | | | | | (w 28) |
| BC | Chronic | None | W 44 | 300 | 589 | 282 | 10,000 | 48,100 |

| | Serum reactivity to HIV-1 Tat during STI | | | | |
|---|---|---|---|---|---|
| | Ab titer (μg/ml)⁴ | | | ETIC Score⁵ | |
| | Samples | | | versus reference | |
| | | Positive Ab | | Samples | |
| Patient | Day 1 | Response | Terminal | Day 1 | Terminal |
| YZ | 109 | + | 9.5 | 3.75 | 3 |
| XZ | 27 | + | 15 | 2.43 | 2 |
| WY¹ | 28 | + | 9 | 2.29 | 4 |
| WX | 20 | + | 2.5 | 2.14 | 1 |
| XY | 41 | + | 9 | 2.29 | Bg |
| WZ | 14 | + | 1.7 | 2.29 | 2 |
| FG | 3.3 | − | 2.7 | Bg | Bg |
| GH¹ | 3.54 | − | 2.5 | 0.85 | 1 |
| AB | 8.5 | − | 4 | 1.29 | Bg |
| BD | 4.2 | − | 3.9 | Bg | Bg |
| DE | 2 | − | Bg | 0.70 | Bg |
| BC | 7.75 | − | 4.2 | 2.14 | Bg |

Table II
¹Patients GH, OO, PP and WY left the STI at week 37, 48, 56 and W 78 respectively for unpredictable but independent non-medical reasons from their HIV infection (death by car, change of home town, travel)
²Stage of infection: Patients at an early stage of infection who started HAART at enrollement (primary) and patients at a more advanced stage already under HAART for years before enrollement (chronic).
^{3a}Time t₀ before the 1^{rst} vaccine administration.
^{3b}Terminal sample last collected sample prior to treatment for HAART patients and between 68 to 96 weeks for HAART-free patients
⁴Ab response: positive titers ≧10 μg/ml (cut off value = 5 fold reference serum) and background (Bg) titers < reference serum (<2 μg) (see methods).
⁵Tat inhibiting capacity (ETIC): Positive score at 2 corresponding to 50% ± reference serum and background (Bg) corresponding to <20% reference serum (see methods)
NA: Non available.

TABLE IIb
Group 2b (patients with DcChol adjuvant)

| | Clinical evolution of HIV-1 infection during STI | | | | | | | |
|---|---|---|---|---|---|---|---|---|
| | | | | CD4 cells/μl | | | Plasma viremia | |
| | | | | Samples | | | (copies/ml) | |
| | | | HAART | Prior to vaccine | | | Samples | |
| | Stage of | | resumption | Pre | Time | STI | Prior to vaccine | STI |
| Patient | infection² | HIV-1 related symptoms | (week) | HAART | t₀^{3a} | Terminal^{3b} | (Pre HAART) | Terminal |
| AC | Chronic | None | W 16 | 259 | 927 | 427 | 71,000 | 256,000 |
| BE | Chronic | None | W 24 | 355 | 645 | 269 | 94,000 | 30,000 |
| CE | Primary | Relapsing amygdalitis | W 52 | NA | 586 | 282 | NA | 301,000 |
| | | (w 8-40) | | | | | | |
| CD | Chronic | Acute angina | W 76 | 300 | 473 | 259 | 5,000 | 1,900 |
| | | with swollen lymph | | | | | | |
| | | nodes + cutaneous | | | | | | |
| | | mycosis | | | | | | |
| | | with eczema | | | | | | |
| | | (w 8-16) | | | | | | |
| FF | Chronic | None | No | 580 | 770 | 581 | 25,000 | 4,000 |
| FH | Chronic | None | No | 672 | 945 | >500 | 148,000 | 60,800 |
| GG | Primary | Conjonctivitis (w 3) | No | 619 | 917 | 666 | 10,000 | 27,000 |
| HH | Chronic | Flue-like syndrome | No | 262 | 1,063 | 616 | 29,000 | 16,000 |
| | | (w 8-12) | | | | | | |

| | Serum reactivity to HIV-1 Tat during STI | | | | |
|---|---|---|---|---|---|
| | Ab titer | | | | |
| | (μg/ml)⁴ | | | ETIC Score⁵ | |
| | Samples | | | versus reference | |
| | | Positive Ab | | Samples | |
| Patient | Day 1 | Response | Terminal | Day 1 | Terminal |
| AC | Bg | − | Bg | 0.5 | Bg |
| BE | Bg | − | Bg | Bg | Bg |
| CE | Bg | − | Bg | Bg | Bg |
| CD | 2 | − | 6 | Bg | Bg |
| FF | Bg | − | Bg | 0.95 | Bg |
| FH | 2 | − | Bg | Bg | 1 |
| GG | Bg | − | Bg | Bg | 2 |
| HH | Bg | − | Bg | Bg | Bg |

TABLE IIc
Group 3 (patients with adjuvant-free Tat Toxoid)

| | Clinical evolution of HIV-1 infection during STI | | | | | |
|---|---|---|---|---|---|---|
| | | | | CD4 cells/μl | | |
| | | | HAART | Samples | | |
| | | HIV-1 related | resumption | Prior to vaccine | | STI |
| Patient | Stage of infection² | symptoms | (week) | Pre HAART | Time t₀^{3a} | Terminal^{3b} |
| MN | Early infected | Acute sinusitis | W 28 | 417 | 639 | 442 |
| | | with swollen lymph | | | | |
| | | nodes (w12) | | | | |
| QQ | Chronic | Disseminated | W 28 | 320 | 872 | 460 |
| | | purpura + Flue- | | | | |
| | | like syndrome | | | | |
| | | (w24) | | | | |
| NP | Chronic | None | W 34 | 627 | 386 | 361 |
| OP | Chronic | None | W 40 | 445 | 747 | 331 |
| MO | Chronic | Thrombopenia | W 72 | 660 | 1,369 | 597 |
| | | (w8) | | | | |
| MP | Early infected | Acute diarrhea | W 72 | 330 | 659 | 301 |
| | | (w20) | | | | |
| NO | Early infected | None | W 86 | 360 | 535 | 248 |
| MM | Early infected | None | No | 612 | 678 | 446 |
| NN | Chronic | Flue-like syndrome | No | 560 | 1,090 | 300 |
| | | (w1) | | | | |
| PP¹ | Early infected | None | No | NA | 921 | 848 (w 56) |
| OO¹ | Early infected | None | No | >500 | 763 | 575 (w 48) |

| | | | Serum reactivity to HIV-1 Tat during | | | | |
|---|---|---|---|---|---|---|---|
| | | | STI | | | | |
| | | | Ab titer | | | | |
| | Plasma viremia | | (μg/ml)⁴ | | | | |
| | (copies/ml) | | Samples | | | | |
| | Samples | | | Positive | | ETIC Score⁵ | |
| | Prior to vaccine | STI | | Ab | | Samples | |
| Patient | (Pre HAART) | Terminal | Day 1 | Response | Terminal | Day 1 | Terminal |
| MN | >750,000 | 485,000 | 38 | + | 15.6 | 2.6 | Bg |
| QQ | NA | 261,000 | Bg | − | 7 | 1.6 | Bg |
| NP | NA | 114,000 | 5.4 | − | Bg | 1.2 | Bg |
| OP | 62,000 | 188,000 | Bg | − | Bg | 0.3 | Bg |
| MO | 120,000 | 109,000 | 18 | + | 6.6 | 3 | Bg |
| MP | >750,000 | 77,000 | 13.6 | + | Bg | 3.6 | Bg |
| NO | 12500 | 29,000 | Bg | − | Bg | 3 | Bg |
| MM | 18700 | 3,050 | 2.44 | − | Bg | 1 | Bg |
| NN | 98,000 | 22,000 | 2.10 | − | Bg | 2.6 | 1 |
| PP¹ | NA | 65,000 | Bg | − | Bg | 0 | 1 |
| OO¹ | >10,000 | 31,600 | 6.35 | − | Bg | 1.3 | 2 |

Disease progression in all STI volunteers, as evaluated by the need of HAART resumption, was inversely correlated with the Ab response to Tat for up to 16 months following vaccine trial. At week 68 of the STI, 10 of 19 volunteers with low anti-Tat Ab titer (<10 μg) resumed HAART while only 1 of 9 Ab responders (Titer>10 μg) required ARV treatment (Fisher's exact: 0.049 and Kaplan-Meier Log-rank test Pr:0.0488 (FIG. 8-1). Subgroup Kaplan-Meier analysis was performed on volunteers with chronic infection to explore whether the clinical stage of HIV-1 infection (recent early or established chronic) biased this result, showed that after 68 weeks STI, 5 of 5 Ab responders remained without treatment whereas 9 of 14 Ab non responders received HAART (Fisher's exact 0.035 and Kaplan-Meier Log-rank test Pr:0.0278) (FIG. 8-2).

Of note, all 9 Ab responders were volunteers who received Tat Toxoid either with or without DcChol adjuvant. Comparative analysis which investigated whether DcChol adjuvant contributed in delaying HAART resumption in these patients led to the conclusion that DcChol adjuvanted compared to non-adjuvanted Tat Toxoid immunization correlated with delayed need for HAART, since 6 of 6 versus 0 of 3 Ab responder volunteers from the adjuvanted and unadjuvanted groups respectively remained off-HAART up to 78 weeks (Fisher's exact: 0.0119). Noteworthy even in vaccinees receiving Tat Toxoid without adjuvant, 2 of the 3 patients with positive Ab titers delayed HAART resumption up to week 72 of the STI (Table IIc). The impact of a positive Ab response in delaying ARV treatment induced by DcChol adjuvanted Tat Toxoid vaccine is further emphasized by subgroup analysis of patients receiving DcChol with (group 1) or without (group 2) Tat Toxoid (Tables IIa and b). In the DcChol subpopulation (n=19) whereas 8 of 13 Ab non responders resumed HAART, all the positive responders, as reported above, remained-off treatment all through the STI (Fisher's exact: 0.036 after 2 years with a Kaplan-Meier Log-rank test Pr:0.0246). When analysis focused only chronic patients (n=14) the correlation between positive Ab response and absence of HAART was still valid over the 24 month study with Kaplan-Meier Log-rank test Pr:0.0369.

Despite initial randomization of patients at the time of vaccination, a retrospective analysis of blood cells and sera collected prior to vaccination was nonetheless conducted to verify that differences of anti-Tat Ab response following vaccination were not the direct consequence of differences in immune status of the volunteers. As described in the methods section, the 9 positive anti-Tat Ab responders did not have evidence of differences in overall immune status at time of vaccination (t₀), as assessed by absence of baseline differences between this subgroup of patients and the 22 other patients under STI concerning CD4 cell count, serum Ab response to HIV-1 antigens including gp120, gag, nef and Tat proteins and serum Tat inhibitory capacity. While subtle undefined individual differences in immune competence of volunteers may likely have had an influence on their immune response to HIV-1 antigens including Tat, these data show equal distribution of patients between anti-Tat AbR+and Ab non R subgroups in reference to their immune parameters. As a consequence positive Ab response of Ab R+patients could not be considered the result of superior immune status.

Following Tat Toxoid Vaccination, Sustained Absence of ARV Treatment was Correlated with Serum Tat Inhibitory Bioactivity.

Sera of the 9 Tat Ab responders strongly inhibited Tat transcriptional activity with a Tat inhibitory capacity (ETIC) score over the cut off for positive response (>2) corresponding to an ETIC index of 50% ±10 reference serum value (Table II; FIG. 8B). The inhibition of Tat transcriptional activity triggered by sera was due to Abs since purified IgG from the serum of the 6 responders inhibited Tat transcriptional activity in a dose-dependent manner (FIG. 9A) and more effectively than purified IgG both from the reference standard serum and from other patients sera tested (FIG. 9B). Finally the antagonistic effect of purified Abs was further supported by their high specific avidity to Tat protein, reaching nanomolar ranges (KD*=4.5±2×10⁻⁹ M and 4±3×10⁻⁹ M) in tested Ab Responders (YZ and XZ patients respectively) but not in non responders (AB, BD and HH patients).

In volunteers from the adjuvanted and unadjuvanted Tat toxoid groups, most individuals developing a positive serum Tat inhibitory capacity following vaccination (ETIC score>2) remained HAART-free (10 of 12 volunteers) up to week 68 of the STI in contrast to individuals whose sera did not exhibit a positive ETIC (2 of 8) (Fisher's exact=0.019). The correlation between positive ETIC responders and absence of HAART remained -significant for the group of chronic patients (n=13) (Fisher's exact: 0.0046). These results could be expected given that positive ETIC score was observed mostly in patients with high Ab titer (9 of 9 Ab responders) rather than in those who did not (3 of 14 Ab non-responders) (Fisher's exact:0.0003).

HAART Resumption was Associated with Lack of Serum Tat Inhibitory Bioactivity.

As shown in Table II, sera collected prior to treatment from all the patients who resumed HAART (15 out of 15), did not exhibit Tat inhibitory capacity, (their ETIC score being at background levels corresponding to an ETIC index<20% reference serum value) (see Materials and Methods section). By contrast, sera collected at terminal stage of the study from a majority of patients off HAART (11 of 16) still exhibited some Tat inhibitory capacity, their ETIC score being higher than reference serum (Table II). The correlation between lack of ETIC and medical need of treatment was statistically highly significant (Fisher's exact<0.0001).

Anti-Tat Ab Decrement in Ab Responders to Tat Toxoid.

During the STI, serum antibody titer and ETIC decreased progressively in all Ab responders (Table II). However, in the 6 DcChol adjuvanted antibody responders, ETIC at terminal stages of the study still remained higher than that of reference serum except serum from patient XY which reached reference levels by week 68. Of importance, concomitant with serum ETIC above or equal to reference serum, clinical status remained stable throughout the STI follow-up lasting 18 months for WY and 24 months for the 5 other patients. None of these 6 antibody responders developed HIV-1 related symptoms of disease progression. Moreover at the end of the STI study HIV-1 levels did not increase and CD4 cell count did not decline compared to the pre-HAART status preceding enrollment into the vaccine trial (Table IIa). These clinical data prompted the decision not to resume ARV treatment in these patients during the 2 year STI.

Of note, during STI, serum anti-Tat Abs titer and ETIC remained low in the volunteers receiving adjuvant alone, except in one subject (CD) whose Abs raised by week 10 up to 10 μg accompanying acute angina and dermatitis but dropped at week 60 before resumption of HAART.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Achour et al, AHGP-30, a synthetic analogue of human immunodeficiency virus (HIV-1) p17, is a target for cytotoxic lymphocytes in HIV-1-infected individuals@ Proc. Natl. Acad. Sci. USA 87:7045-7049 (1990)
Akerstrom, B. & Bjorck, L. A physicochemical study of protein G, a molecule with unique immunoglobulin G-binding properties. J Biol Chem. 261, 10240-10247 (1986)
Borrow et al, AAntiretroviral pressure exerted by HIV-1-specific cytotoxic T lymphocytes (CTLs) during primary infection demonstrated by rapid selection of CTL escape virus@ Nat. Med. 3:205-211 (1997)
Butto, S. et al. Tat Multicentric Study Group. Sequence conservation and antibody cross-recognition of clade B human immunodeficiency virus (HIV) type 1 Tat protein in HIV-1-infected Italians, Ugandans, and South Africans. J Infect Dis. 188, 1171-1180. Epub Sep. 30, 2003 (2003).
Cafaro, A. et al. Control of SHIV-89.6P-infection of cynomolgus monkeys by HIV-1 Tat protein vaccine. Nat Med. 5, 643-650 (1999).
Calarota, S. A. et al. Immune responses in asymptomatic HIV-1-infected patients after HIV-DNA immunization followed by highly active antiretroviral treatment. J Immunol. 163, 2330-2338 (1999).
Caputo, A., Gavioli, R. & Ensoli, B. Recent advances in the development of HIV-1 Tat-based vaccines. Curr HIV Res. 2, 357-356 (2004).
Chirmule et al, AHuman immunodeficiency virus tat induces functional unresponsiveness in T cells@ J. Virol. 69:492-498 (1995)
Cullen, ARegulation of HIV-1 gene expression@ FASEB J. 5:2361-2368 (1991)
Derdeyn, C. A. et al. Sensitivity of human immunodeficiency virus type 1 to the fusion inhibitor T-20 is modulated by coreceptor specificity defined by the V3 loop of gp120. J Virol. 74, 8358-8367 (2000).
Ensoli, B. et al. Release, uptake, and effects of extracellular human immunodeficiency virus type 1 Tat protein on cell growth and viral transactivation. J Virol. 67, 277-287 (1993).
Ensoli, B., Barillari, G., Salahuddin, S. Z., Gallo, R. C. & Wong-Staal, F. Tat protein of HIV-1 stimulates growth of cells derived from Kaposi's sarcoma lesions of AIDS patients. Nature. 345, 84-86 (1990).
Felber, B. K. & Pavlakis, G. N. A quantitative bioassay for HIV-1 based on trans-activation. Science 239, 184-187 (1988)
Finkel, T. H. et al. Apoptosis occurs predominantly in bystander cells and not in productively infected cells of HIV- and SIV-infected lymph nodes. Nat Med. 1, 129-134 (1995).
Gallo et al, AFrequent detection and isolation of cytopathic retroviruses (HTLV-III) from patients with AIDS and at risk of AIDS@ Science 224:500-503 (1984)
Gallo, R. C. Tat as one key to HIV-induced immune pathogenesis and Tat (correction of Pat) toxoid as an important component of a vaccine. Proc Natl Acad Sci USA. 96, 8324-8326 (1999).
Gallo, R. C. The end or the beginning of the drive to an HIV-preventive vaccine: a view from over 20 years. Lancet online September 2005.
Gougeon, M. L. Apoptosis as an HIV strategy to escape immune attack. Nat Rev Immunol. 3:392-404 (2003).
Goulder et al, A Late escape from an immunodominant cytotoxic T-lymphocyte response associated with progression to AIDS@ Nat. Med. 3:212-217 (1997)
Goulder et al., A Novel, cross-restricted, conserved, and immunodominant cytotoxic T lymphocyte epitopes in slow/non-progressors in HIV-1 type 1 infection@ AIDS Res. Hum. Retroviruses 12:1691-1698 (1996)
Gray, D, & Skarvall, H. B-cell memory is short-lived in the absence of antigen. Nature. 336, 70-73. (1988).
Gringeri et al, ASafety and immunogenicity of HIV-1 tat toxoid in immunocompromised HIV-1-infected patients@ J. of Human Virology (1998)
Gringeri, A. et al. Tat toxoid as a component of a preventive vaccine in seronegative subjects. J Acquir Immune Defic Syndr Hum Retrovirol. 20, 371-375 (1999).
Gringeri, A. et al. Safety and immunogenicity of HIV-1 Tat toxoid in immunocompromised HIV-1-infected patients. J Hum Virol. 1, 293-298 (1998).
Gutheil, W. G. et al. Human immunodeficiency virus 1 Tat binds to dipeptidyl aminopeptidase IV (CD26): a possible mechanism for Tat's immunosuppressive activity. Proc Natl Acad Sci USA. 91, 6594-6598 (1994).
Hendel et al, AContribution of cohort studies in understanding HIV-1 pathogenesis: introduction of the GRIV cohort and preliminary results@ BioMedicine and Pharmacol. 50:480-487 (1996)
Hermans, Ph. & Clumeck, N. Safety and immunogenecity of an HIV-1 Tat toxoid vaccine in HIV-1 infected volunteers on HAART. Abstract 644. 10th Conference on Retroviruses and Opportunistic Infection (CROI) Boston (2003).
Ho, D. D. et al. Rapid turnover of plasma virions and CD4 lymphocytes in HIV-1 infection. Nature 373, 123-126 (1995).
chi.ucs2.edu/vaccinees
Hynes, AIntegrins: a family of cell surface receptors@ Cell 48:549-554 (1987)
Hynes, AIntegrins: versatility, modulation, and signaling in cell adhension@ Cell 69:11-25 (1992)
Katsikis et al, AHIV-1 type 1 tat protein enhances activation but not Fas (CD95) induced peripheral blood T cell apoptosis in healthy individuals@ Int. Immunol. 9:835-841 (1997)
Krone et al., ANatural antibodies to HIV-1-tat epitopes and expression of HIV-1 genes in vivo® J. Med. Virol. 26:261-270 (1988)
Le Buanee et al., AProphylactic and therapeutic AIDS vaccine containing as a component the innocuous Tat toxoid® Biomed Pharm/AIDS Sci. 52:431-5 (1998)
Li et al, AInduction of apoptosis in uninfected lymphocytes by HIV-1 tat protein@ Science 268:429-431 (1995)
Li et al, Atat protein induces self-prepetuating permissivity for productive HIV-1 infection@ Proc. Natl. Acad. Sci. USA 93:14720-14725 (1997)
Liang, X. et al. Vectored Gag and Env but not Tat show efficacy against simian-human immunodeficiency virus 89.6P challenge in Mamu-A*01-negative rhesus monkeys. J Virol. 79, 12321-12331 (2005)
Lolli et al., AEffect of antibody to HIV-1 tat protein on viral replication in vitro and progression of HIV-1 disease in vivo@ J. Acquir. Immune Defic. Syndr. Hum. Retrovirol @ 10:408-416 (1995)
Marcuzzi, A., Weinberger, J. & Weinberger, O. K. Transcellular activation of the human immunodeficiency virus type 1 long terminal repeat in cocultured lymphocytes. J Virol. 66, 4228-4232 (1992).
McCloskey, T. W. et al. Dual role of HIV Tat in regulation of apoptosis in T cells. J Immunol. 158, 1014-1019 (1997).
Nardelli, B., Gonzalez, C. J., Schechter, M. & Valentine, F. T. CD4⁺ blood lymphocytes are rapidly killed in vitro by contact with autologous human immunodeficiency virus-infected cells. Proc Natl Acad Sci USA. 92, 7312-7316 (1995).
Nixon et al, ACTL reconition of HIV-1 proteins and peptides@ AIDS 5:1055-1056 (1991)
Okamoto, T., Benter, T., Josephs, S. F., Sadaie, M. R. & Wong-Staal, F. Transcriptional activation from the long-terminal repeat of human immunodeficiency virus in vitro. Virology 177, 606-614 (1990).
Osterhaus, A. D., et al. Vaccination with Rev and Tat against AIDS. Vaccine. 17, 2713-2714 (1999).
Picard et al, AA 2-year follow-up of an anti-HIV-1 immune reaction in HIV-1 gp160-immunized healthy seronegative humans: evidence for persistent celll-mediated immunity@ JAIDS 5:539-546 (1992)
Pauza, C. D. et al. Vaccination with tat toxoid attenuates disease in simian/HIV-challenged macaques. Proc Natl Acad Sci USA. 97, 3515-3519 (2000).
Rappaport et al, A32 bp CCR5 gene deletion and resistance to fast progression among HIV-1 infected heterozygotes@ The Lancet 349:922-923 (1997)
Re, M. C. et al. Antibodies against full-length Tat protein and some low-molecular-weight Tat-peptides correlate with low or undetectable viral load in HIV-1 seropositive patients. J Clin Virol. 21, 81-89 (2001).
Re, M. C. et al. Effect of antibody to HIV-1 Tat protein on viral replication in vitro and progression of HIV-1 disease in vivo. J Acquir Immune Defic Syndr Hum Retrovirol. 10, 408-416 (1995).
Reiss et al., A Contribution of antibody response to recombinant HIV-1 gene-encoded products nef, rev, tat, and protease in predicting development of AIDS in HIV-1-infected individuals@ J. Acquir. Immune Defic. (1991)
Rezza, G. et al. The presence of anti-Tat antibodies is predictive of long-term nonprogression to AIDS or severe immunodeficiency: findings in a cohort of HIV-1 seroconverters. J Infect Dis. 191, 1321-1324 (2005).
Shi et al., AApoptosis induced by HIV-1 Tat protein and TNF-alpha: Potentiation of neurotoxicity mediated by oxidative stress and implications for HIV-1 dementia@ J. Neuroviral 4:281-90 (1998)
Viscidi et al, A Inhibition of antigen-induced lymphocyte proliferation by tat protein from HIV-1″ Science 246:1606-1608 (1989)
Viscidi, R. P., Mayur, K., Lederman, H. M. & Frankel, A. D.

Inhibition of antigen-induced lymphocyte proliferation by Tat protein from HIV-1. Science 246, 1606-1608 (1989).
Voss, G. et al. Prevention of disease induced by a partially heterologous AIDS virus in rhesus monkeys by using an adjuvanted multicomponent protein vaccine. J Virol. 77, 1049-1058 (2003).
Weeks et al, AIdentification of a novel cell attachment domain in the HIV-1 tat protein and its 90-kDa cell surface binding protein@ J. Biol. Chem. 268:5279-5284 (1993)
Wei, X. et al. Viral dynamics in human immunodeficiency virus type 1 infection. Nature 373, 117-122 (1995).
Westendorp et al, ASensitization of T cells to CD95-mediated apoptosis by HIV-1 tat and gp120″ Nature 375:497-500 (1995)
Westendorp, M. O. et al. HIV-1 Tat potentiates TNF-induced NF-kappa B activation and cytotoxicity by altering the cellular redox state. EMBO J. 14, 546-554 (1995).
Westendorp, M. O. et al. Sensitization of T cells to CD95-mediated apoptosis by HIV-1 Tat and gp120. Nature 375, 497-500 (1995).
Westendorp, M. O., Li-Weber, M., Frank, R. W. & Krammer, P. H. Human immunodeficiency virus type 1 Tat upregulates interleukin-2 secretion in activated T cells. J Virol. 68, 4177-4185 (1994).
Wong-Staal, F. & Haseltine, W. A. Regulatory genes of human immunodeficiency viruses. Mol Genet Med. 2,. 189-219 (1992).
Zagury et al, AC-C chemokines, pivotal in protection against HIV-1 infection@ Proc. Natl. Acad. Sci. USA 1998b (in press) Zagury et al, AIFNα and tat involvement in the immunosuppression of uninfected T cells and C-C chemokine decline in AIDS@ Proc. Natl. Acad. Sci. USA 1998a (in press)
Zagury et al, ALong-term cultures of HTLV-III-infected T cells: a model of cytopathology of T-cell depletion in AIDS@ Science 231:850-853 (1986)
Zagury et al, AModel of AIDS immunopathogenesis based on the HIV-1 gp120 and tat-induced dysregulation of uninfected immune cells@ Cellular Pharmacology 3:123-128 (1996)
Zagury, AA naturally unbalanced combat@ Nat. Med. 3:156-157 (1997)
Zagury, D. et al. Interferon alpha and Tat involvement in the immunosuppression of uninfected Tcells and C-C chemokine decline in AIDS. Proc Natl Acad Sci USA. 95, 3851-3856 (1998).
Zagury, J. F. et al. Antibodies to the HIV-1 Tat protein correlated with nonprogression to AIDS: a rationale for the use of Tat toxoid as an HIV-1 vaccine. J Hum Virol. 1, 282-292 (1998).
Zauli, G. et al. Tat protein stimulates production of transforming growth factor-beta 1 by marrow macrophages: a potential mechanism for human immunodeficiency virus-1-induced hematopoietic suppression. Blood. 80, 3036-3043 (1992).
Zhang et al, AProtective immunity to HIV-1 in SCID/beige mice reconstituted with peripheral blood lymphocytes of exposed but uninfected individuals@ Proc. Natl. Acad. Sci. USA 93:14725-14725 (1996)
Zhang, Z., Vuori, K., Reed, J. C. & Ruoslahti, E. The alpha 5 beta 1 integrin supports survival of cells on fibronectin and up-regulates Bcl-2 expression. Proc Natl Acad Sci USA. 92, 6161-6165 (1995).

What is claimed is:

1. A method of monitoring anti-HIV treatment in a subject infected with HIV, the method comprising:
(i) assessing the presence of anti-tat antibodies in a sample derived from said subject, and
(ii) assessing the presence of a neutralizing anti-tat activity in a sample derived from said subject having anti-tat antibodies,
the presence of a neutralizing anti-tat activity being indicative of the required treatment protocol.

2. The method of claim 1, wherein the absence of anti-tat antibody or neutralizing anti-tat activity in said subject is an indication that the anti-HIV treatment shalt be maintained or resumed.

3. The method of claim 1, wherein the presence of anti-tat antibody or a neutralizing anti-tat activity in said subject is an indication that the treatment may be interrupted or that the treatment interruption may be prolonged.

4. A method of monitoring anti-HIV treatment in an infected HIV subject under structured treatment interruption, the method comprising:
(i) assessing the presence of anti-tat antibodies in a sample derived from said subject, and
(ii) assessing the presence of a neutralizing anti-tat activity in a sample derived from said subject having anti-tat antibodies,
the presence of a neutralizing anti-tat activity being an indication that the treatment interruption may be prolonged.

5. The method of claim 4, wherein the anti-HIV treatment in said subject having a neutralizing anti-tat activity is subsequently monitored by assessing the presence of anti-tat antibodies in a sample derived from said subject.

6. A method of monitoring anti-HIV treatment in an HIV infected subject under structured treatment interruption, wherein the subject has been previously shown to have a neutralizing anti-tat activity, the method comprising assessing the presence of anti-tat antibodies in a sample derived from said subject, wherein the presence of anti-tat antibodies in said subject is an indication that the treatment interruption may be prolonged.

7. The method of claim 6, wherein the anti-HIV treatment comprises HAART.

8. The method of claim 6, wherein the subject has received an anti-Tat vaccine, in addition to said anti-HIV treatment.

9. The method of claim 8, wherein the anti-tat vaccine is a Tat Toxoid.

10. The method of claim 4, wherein the anti-HIV treatment comprises HAART.

11. The method of claim 4, wherein the subject has received an anti-Tat vaccine, in addition to said anti-HIV treatment.

12. The method of claim 11, wherein the anti-tat vaccine is a Tat Toxoid.

13. The method of claim 4, wherein the sample is a sample of blood or serum derived from said subject.

14. The method of claim 4, wherein the level of antibody or neutralizing anti-tat activity measured with the sample is compared to a mean value or to the level of antibody or neutralizing anti-tat activity of a reference sample, respectively.

15. The method of claim 14, wherein a significant anti-tat antibody level or a significant neutralizing anti-tat activity is a level or activity, respectively, that is at least twice superior to the mean value or reference sample.

16. The method of claim 4, wherein the level of anti-tat antibody is measured by an immunological or immuno-enzymatic assay.

17. The method of claim 4, wherein the level of neutralizing anti-tat activity is measured by contacting a tat-responsive reporter gene in the presence of Tat and the sample derived from the subject, and determining the level of expression of the reporter gene.

18. The method of claim 17, further comprising comparing the level of expression of the reporter gene as measured with the test serum or blood sample, to the level of expression of the reporter gene as measured in the presence of a reference serum or blood sample.

19. The method of claim 4, wherein the level of antibody or neutralizing anti-tat activity is assessed at different times.

20. A method of prolonging anti-HIV treatment interruption in a HIV infected subject, the method comprising administering to the subject an anti-Tat vaccine.
